(19) United States
(12) Patent Application Publication
(10) Pub. No.: US 2026/0166147 A1
(43) Pub. Date: Jun. 18, 2026

(54) T CELL CIRCUITS FOR REDUCING BRAIN INFLAMMATION

(71) Applicant: The Regents of the University of California, Oakland, CA (US)

(72) Inventors: Milos SIMIC, San Francisco, CA (US); Nishith REDDY, San Francisco, CA (US); Wendell A. LIM, San Francisco, CA (US)

(21) Appl. No.: 19/126,186

(22) PCT Filed: Nov. 2, 2023
(86) PCT No.: PCT/US2023/078555

§ 371 Date: Apr. 30, 2025

Related U.S. Application Data
(60) Provisional application No. 63/422,817, filed on Nov. 4, 2022.
(60) Provisional application No. 63/453,688, filed on Mar. 21, 2023.

(51) Int. Cl. A61K 40/42; A61K 38/18; A61K 38/20; A61K 40/11; A61K 40/31; A61K 40/41; A61P 37/06

(52) U.S. Cl. CPC A61K 40/4254; A61K 38/1841; A61K 38/2066; A61K 40/11; A61K 40/31; A61K 40/416; A61P 37/06

(57) ABSTRACT

This disclosure provides, among other things, an engineered immunosuppressive (e.g., CD4+) T cell comprising a molecular circuit comprising the following components: (a) a central nervous system (CNS)-specific binding-triggered transcriptional switch (BTTS); and (b) a nucleic acid encoding an anti-inflammatory payload, wherein binding of the BTTS to a marker on the surface of a target cell activates expression of the anti-inflammatory payload by the cell. A method of reducing inflammation in the brain of a subject is also provided.

### CROSS-REFERENCING

This application claims the benefit of U.S. provisional patent application Ser. No. 63/422,817 filed on Nov. 4, 2022, and U.S. provisional patent application Ser. No. 63/453,688, filed on Mar. 21, 2023, which applications are incorporated by reference herein.

### INCORPORATION BY REFERENCE OF SEQUENCE LISTING PROVIDED AS A SEQUENCE LISTING XML FILE

A Sequence Listing is provided herewith as a Sequence Listing XML, “UCSF-704WO_SEQ_LIST”, created on Oct. 27, 2023, and having a size of 9,700 bytes. The contents of the Sequence Listing XML are incorporated herein by reference in their entirety.

### INTRODUCTION

Chronic inflammatory diseases represent one of the most significant causes of death worldwide. A major challenge is to develop therapies that suppress inflammation in a location-specific manner, without causing systemic immune suppression. This is a particular challenge for neuroinflammatory diseases such as MS, the most common inflammatory and demyelinating disorder of the CNS. While B cell depleting therapies have shown great improvement in relapsing forms of MS, they do not resolve progressive MS, including chronic active lesions which are histologically comprised of T cells and activated macrophages/microglia foci, suggesting that broader approaches are needed.

Numerous immunotherapeutic molecules (cytokines, chemokines, antibodies) have been identified as having therapeutic potential in preclinical models for MS, such as experimental autoimmune encephalomyelitis (EAE). However, the translation of these findings in human clinical trials has been disappointing, often due to insufficient penetration of the molecule into the CNS (inadequate crossing of the BBB) or off-target systemic toxicity due to the pleiotropic effects of cytokines and other immunomodulatory factors. A good example is the cytokine interleukin-10 (IL-10), a potent anti-inflammatory molecule. Clinical trials testing IL-10 infusions were discontinued due to lack of efficacy, likely due to its short half-life (˜3 h in both humans and mice) and inability to cross the BBB. In contrast, however, direct expression of IL-10 via adenovirus injected directly into the CNS did ameliorate disease.

There is therefore a great need for better treatments for inflammatory disorders of the brain.

### SUMMARY

This disclosure provides, among other things, programmable brain-sensing T cells engineered to locally deliver therapeutic payloads that are customized for neuroinflammation. A set of CNS-specific extracellular ligands were identified, antibodies against these were screened for, and used to create CNS-activated synthetic binding-triggered transcriptional switches (i.e., engineered receptors that sense an extracellular antigen and respond by inducing a transcriptional response). This platform was then used to engineer CNS-induced T cells that locally produce genetically-encoded payloads for treatment of a variety of inflammatory CNS diseases. CNS-induced expression of immunosuppressive payloads, such as the cytokine IL-10, can ameliorate neuroinflammation in experimental autoimmune encephalomyelitis (EAE), a mouse model of multiple sclerosis (MS). This tissue-targeted cell platform provides dual-levels of targeting specificity: production of the therapeutic payload is restricted only to the tissue of interest (here the CNS), but the payload itself also has its own intrinsic molecular targeting specificity within this tissue. Such a local delivery strategy thereby avoids potential toxic systemic cross-reactions of the molecular payload in other non-disease tissues.

In some embodiments, the cell may be an engineered CD4+ T cell comprising a molecular circuit comprising the following components: (a) a central nervous system (CNS)-specific binding-triggered transcriptional switch (BTTS); and (b) a nucleic acid encoding an anti-inflammatory payload, wherein binding of the BTTS to a marker on the surface of a target cell activates expression of the anti-inflammatory payload by the cell. A method of reducing inflammation in the brain of a subject is also provided. In any embodiment, the payload can be IL-10 or TGFβ.

In some embodiments, the BTTS may recognizes a CNS-specific cell surface marker such as MOG, CDH10, CSPG5, PTPRZ1, BCAN, GRM3, Neurexin 1b and NrCAM. In any embodiments, the payload may be i. an anti-inflammatory cytokine, ii. an antibody that blocks pro-inflammatory signaling, iii. a soluble receptor that binds to pro-inflammatory cytokine, iv. a cytokine sink or any combination thereof (e.g., i and ii, i and iii, i and iv, ii and iii, ii and iv, iii and iv, i, ii and iii, i, iii and iv, ii, iii and iv or i, ii, iii and iv). In some embodiments, binding of the BTTS to the CNS-specific antigen may activate expression of TGFβ1, CD25 and, optionally, IL-10 or a variant thereof although other combinations could be used.

For example, in any embodiment the BTTS may be activated by binding to BCAN and the anti-inflammatory payload may comprise IL-10. In any embodiment the BTTS may be activated by binding to CDH10 and the anti-inflammatory payload may comprise IL-10. In any embodiment, the BTTS may be activated by binding to BCAN and the anti-inflammatory payload may comprise TGFβ. In any embodiment, the BTTS may be activated by binding to CDH10 and the anti-inflammatory payload may comprise TGFβ. In any of these embodiments, the anti-inflammatory payload may further comprise CD25 and/or IL-2 or a variant thereof.

Also provided is a method of reducing brain inflammation in a subject. This method may comprise administering the engineered CD4+ T cell to the subject. In some cases, the subject may have an inflammatory brain disease, e.g., multiple sclerosis or encephalitis, etc. In these embodiments, the cells may be used as part of a treatment for the disease.

Using a cell to selectively and autonomously deliver therapeutic payloads to the brain could, in principle, reduce systemic off-target toxicity while also increasing local efficacy. For example, in the case of chronic inflammation, such as MS, systemic treatment with anti-inflammatory drugs can result in increased risk of infections and other pathologies. Immune cells have evolved to infiltrate diverse tissues, respond to injury or infection, and reshape tissue ecosystems, properties that may make them suitable to act as local therapeutic delivery agents. T cells have the ability to cross the blood brain barrier (BBB). One way to harness T cells to deliver payloads selectively to the brain would be to engineer them to recognize non-disease CNS-specific antigens, and to use these to trigger production of an anti-inflammatory payload. A cell-based CNS-specific delivery platform could yield a common disease-agnostic platform to treat numerous CNS diseases.

Having a therapeutic cell produce a therapeutic biologic in the target tissue should help with efficacy. For example, many cytokines, such as IL-10, have a short biological half-life (˜3 h). Locally producing these factors could overcome these issues of pharmacokinetics. Such tissue targeted cells can also be combined with genetically modified biologics that have specific improved properties. In addition, brain-sensing therapeutic cells could help significantly with the specific challenges associated with delivering many therapeutic agents across the BBB. Brain-sensing cells could be used as a platform to treat a broad set of CNS diseases including neuroinflammation or even neurodegeneration.

Examples of such circuits, their use and further advantages of the same may be described in further detail below.

### BRIEF DESCRIPTION OF THE DRAWINGS

The skilled artisan will understand that the drawings, described below, are for illustration purposes only. The drawings are not intended to limit the scope of the present teachings in any way.

FIG. 1. shows synNotch induced production of suppressive cytokine TGFβ.

FIG. 2. shows that suppressor cells that produce combination of TGFβ (suppressive cytokine) and CD25 (IL2 sink) are very effective at suppressing CAR T killing in vitro.

FIG. 3. shows that suppressor cells that produce combination of IL10 (suppressive cytokinc) and CD25 (IL2 sink) are very effective at suppressing CAR T killing in vitro.

FIG. 4. shows that suppressor cells that produce combination of TGFβ (suppressive cytokine) and CD25 (IL2 receptor) are very effective at suppressing CAR T killing of tumors in vivo.

FIG. 5. shows that engineered T cells overexpressing CD25 increases consumption of IL2 and cell proliferation. CD25 should increase survival/persistence of the T cells in the host.

FIG. 6. shows that synNotch→IL10 synthetic suppressor cells can block autoimmune cell proliferation in brain and CNS in mouse neuroinflammation model.

FIG. 7 illustrates a strategy for inhibiting pathogenic autoreactive T cells to reduce their pathogenicity.

FIG. 8 primary human T cells were engineered to express therapeutic payloads such as the ones described in the top panel, using a CNS-specific synNotch. These cells work as expected as shown by intracellular stain and by ELISA, as shown later.

FIG. 9 generally describes the in vitro assay used to determine if engineered human T cells can inhibit the proliferation of MOG autoreactive mouse T cells.

FIG. 10 cells containing a CDH10 synNotch construct were tested for inhibition of proliferation of the MOG autoreactive mouse T cells by delivering an anti-inflammatory protein such as IL10 or TGFb using the assay of FIG. 9. The results show proliferation can be inhibited by the cells. Depending on the ratios of the autoreactive mouse T cells and the therapeutic T cells, similar levels of inhibition can be achieved as MOG reactive regulatory mouse T cells (see for example ratio 1:8) as estimated by the division index. This data shows that engineered human CD4 T cells can inhibit proliferation of MOG autoreactive mouse T cells.

FIG. 11 illustrates an EAE model.

FIG. 12 shows the EAE scoring system, which was used to clinically evaluate the test animals.

FIG. 13 shows results from an assay designed to test whether the human CDH10 synNotch-IL10 circuit can reduce decrease disease severity.

FIG. 14A: Brain-specific synNotch circuits can be programmed to produce anti-inflammatory cytokine IL-10. CNS-specific synNotch cell can be used to modulate neuroinflammation using for example the CNS-specific marker CDH10 as a priming antigen to deliver IL10, a potent anti-inflammatory cytokine.

FIGS. 14B and 14C. Primary human T cells were engineered with α-CDH10 synNotch-mIL10 circuit to specifically express IL10 upon recognition of CDH10+ cells, in 14B. intracellular staining of IL10 shows specific expression of IL10 only when primary human T cells are cultured with CDH10+K562s (representative of n=3), and in 14C. Quantification of IL10 by ELISA shows specific section of IL10 in only when primary human T cells as cultured with CDH10+K562s (n=3). Unpaired T-test.

FIG. 14D. Schematic of the inhibition of proliferation assay: conventional CD25-MOG TCR CD4 T cells were sorted from the 2D2 MOG TCR transgenic mice. Conventional MOG TCR autorcactive T cells were stained with a membrane stain (celltrace violet) which allows quantification of the T cell division by analyzing the dilution of the dye at each cell division when cocultured with APC presenting MOG peptide. To assay the inhibition of proliferation, MOG TCR autoreactive T cells were cocultured at an 8:1 ratio with control BFP, therapeutic T cells α-CDH10 synNotch-IL10 or MOG TCR regulatory T cells for 5 days in presence of K562 CDH10 expressing cells to induce payload expression.

FIG. 14E. Cells were then analyzed by flow cytometry to evaluate the extent of membrane dye dilution and the division index was quantified with the software FlowJo. n=2-3, mean+/−std, 2-way ANOVA followed by multiple comparison test. *=p<0.05

FIGS. 15A-15C show that CNS-specific synNotch circuits can be programmed to produce anti-inflammatory cytokine IL-10.

FIG. 15A: CNS-specific synNotch cells could in principle be used to modulate neuroinflammation. For example, CNS-priming could be used to trigger expression of IL-10, a potent anti-inflammatory cytokine. For more analysis of optimal CNS antigens to target in neuroinflammation.

FIG. 15B: Primary human T cells were engineered with α-BCAN synNotch-mIL→10 circuit and co-cultured with K562 cells engineered to express BCAN. Supernatant was collected after 48 h and IL10 was quantified by ELISA. Quantification shows the specific secretion of IL-10 only when the engineered T cells are cultured with BCAN⁺ K562 cells (n=3).

FIG. 15C: In vitro inhibition assays of microglia and T cell activation. BV2 mouse microglia were cultured with control or therapeutic T cells (α-BCAN synNotch→IL-10) in the presence of BCAN⁺ K562 cells to induce payload expression. 2 hours later, IFN-γ and LPS were added to induce activation of the microglia. Cells were cultured for 24 h and the supernatant was collected to assess inflammation by assaying for secretion of IL-6 and TNF-α by ELISA. n=3, mean+/−std. Conventional CD25⁻ TCR⁺ CD4⁺ T cells were sorted from MOG-specific TCR (2D2) transgenic mice and co-cultured with APCs presenting MOG peptide to induce their activation. To assay the inhibition of activation, MOG-specific TCR 2D2 CD4 T cells were co-cultured at a 1:1 ratio with control transduced or engineered with α-BCAN synNotch→IL-10 T cells for 4 days in presence of BCAN⁺ K562 cells (to stimulate IL-10 induction). Activation of the MOG-specific TCR 2D2 CD4 T cells was analyzed by flow cytometry using the activation marker CD25, or by ELISA to measure IFN-γ secretion (n=3, mean+/−std).

FIGS. 16A-16C shows that a CNS-targeted anti-inflammatory circuit ameliorates autoimmune encephalomyelitis model.

FIG. 16A: Schematic of adoptive transfer EAE model: RAG-1 −/− mice received an adoptive transfer of Th17 polarized CD4 T cells (20×10⁶ in 16B and 25×10⁶ in 16C) from P35-55 MOG immunized C57BL/6J mice. At indicated days post adoptive transfer (arrows), mice received primary human CD4 T cells transduced with either control (no circuit, n=5) or α-BCAN synNotch→IL10 circuit (n=5) at the indicated times (10×10⁶). The EAE neurological disease scoring scale is shown.

FIG. 16B: Treatment with α-BCAN synNotch-IL10 T cells yields improved EAE scores and increased survival. 10⁶ T cells were injected on each day indicated by a black arrow. A 2-way ANOVA was performed, p<0.05. EAE severity was assessed by the area under the curve of each animal starting from the day of first treatment day 7 until day 25. n=5, mean+/−standard error. Unpaired T-test, p<0.05. Survival curves shows improved protection by the α-BCAN synNotch→IL10 T cell treatment as analyzed by log-rank (Mantel-Cox) test.

FIG. 16C: Treatment with α-CDH10 synNotch-IL10 T cells yields improved EAE scores and increased survival. 10⁶ T cells were injected on each day indicated by a black arrow. EAE scores showed significant improvements with CNS-targeted α-CDH10 synNotch→IL10 T cells. A 2-way ANOVA was performed, p<0.05. EAE severity was assessed by the area under the curve of each animal starting from the day of first treatment day 7 until day 25. n=7, mean+/−standard error. Unpaired T-test, p<0.05. Survival curves shows improved protection by the α-CDH10 synNotch→IL10 T cell treatment as analyzed by log-rank (Mantel-Cox) test.

### DEFINITIONS

As used herein, the terms “treatment,” “treating,” “treat” and the like, refer to obtaining a desired pharmacologic and/or physiologic effect and/or a response related to the treatment. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

A “therapeutically effective amount” or “efficacious amount” refers to the amount of an agent (including biologic agents, such as cells), or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated. The terms “individual,” “subject,” “host,” and “patient,” used interchangeably herein, refer to a mammal, including, but not limited to, murines (e.g., rats, mice), non-human primates, humans, canines, felines, ungulates (e.g., equines, bovines, ovines, porcines, caprines), lagomorphs, etc. In some cases, the individual is a human. In some cases, the individual is a non-human primate. In some cases, the individual is a rodent, e.g., a rat or a mouse. In some cases, the individual is a lagomorph, e.g., a rabbit.

As used herein, the term “binding-triggered transcriptional switch” or “BTTS” refers to any polypeptide or complex of the same that is capably of transducing a specific binding event on the outside of the cell (e.g., binding of an extracellular domain of the BTTS) to activation of a recombinant promoter within the nucleus of the cell. Many BTTSs work by releasing a transcription factor that activates the promoter. In these embodiments, the BTTS is made up of one or more polypeptides that undergo proteolytic cleavage upon binding to the antigen to release a gene expression regulator that activates the recombinant promoter. For example, a BTTS may comprise (i) an extracellular domain comprising the antigen binding region of an antigen-specific antibody; (ii) a proteolytically cleavable sequence comprising one or more proteolytic cleavage sites; and (iii) an intracellular domain, wherein binding of the antigen binding region to the antigen induces cleavage of the sequence at the one or more proteolytic cleavage sites, thereby releasing the intracellular domain and wherein the intracellular domain activates transcription of an expression cassette. A BTTS can be based on synNotch, A2, MESA, or force receptor, for example, although others are known or could be constructed.

The terms “synthetic”, “chimeric” and “engineered” as used herein generally refer to artificially derived polypeptides or polypeptide encoding nucleic acids that are not naturally occurring. Synthetic polypeptides and/or nucleic acids may be assembled de novo from basic subunits including, e.g., single amino acids, single nucleotides, etc., or may be derived from pre-existing polypeptides or polynucleotides, whether naturally or artificially derived, e.g., as through recombinant methods. Chimeric and engineered polypeptides or polypeptide encoding nucleic acids will generally be constructed by the combination, joining or fusing of two or more different polypeptides or polypeptide encoding nucleic acids or polypeptide domains or polypeptide domain encoding nucleic acids. Chimeric and engineered polypeptides or polypeptide encoding nucleic acids include where two or more polypeptide or nucleic acid “parts” that are joined are derived from different proteins (or nucleic acids that encode different proteins) as well as where the joined parts include different regions of the same protein (or nucleic acid encoding a protein) but the parts are joined in a way that does not occur naturally.

The term “recombinant”, as used herein describes a nucleic acid molecule, e.g., a polynucleotide of genomic, cDNA, viral, semisynthetic, and/or synthetic origin, which, by virtue of its origin or manipulation, is not associated with all or a portion of the polynucleotide sequences with which it is associated in nature. The term recombinant as used with respect to a protein or polypeptide means a polypeptide produced by expression from a recombinant polynucleotide. The term recombinant as used with respect to a host cell or a virus means a host cell or virus into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

The term “operably linked” refers to a juxtaposition wherein the components so described are in a relationship permitting them to function in their intended manner. For instance, a promoter is operably linked to a coding sequence if the promoter affects its transcription or expression. Operably linked nucleic acid sequences may but need not necessarily be adjacent. For example, in some instances a coding sequence operably linked to a promoter may be adjacent to the promoter. In some instances, a coding sequence operably linked to a promoter may be separated by one or more intervening sequences, including coding and non-coding sequences. Also, in some instances, more than two sequences may be operably linked including but not limited to e.g., where two or more coding sequences are operably linked to a single promoter.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

A “vector” or “expression vector” is a replicon, such as plasmid, phage, virus, or cosmid, to which another DNA segment, i.e. an “insert”, may be attached so as to bring about the replication of the attached segment in a cell.

The term “heterologous”, as used herein, means a nucleotide or polypeptide sequence that is not found in the native (e.g., naturally-occurring) nucleic acid or protein, respectively. Heterologous nucleic acids or polypeptide may be derived from a different species as the organism or cell within which the nucleic acid or polypeptide is present or is expressed. Accordingly, a heterologous nucleic acids or polypeptide is generally of unlike evolutionary origin as compared to the cell or organism in which it resides.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cell” includes a plurality of such cells and reference to “the cell” includes reference to one or more cells and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

As summarized above, the present disclosure provides a engineered CD4+ T cell comprising a molecular circuit comprising the following components: (a) a central nervous system (CNS)-specific binding-triggered transcriptional switch (BTTS) (e.g., a BTTS that recognizes MOG, CDH10, CSPG5, PTPRZ1, BCAN, GRM3, Neurexin 1b and NrCAM) and (b) a nucleic acid encoding an anti-inflammatory payload, wherein binding of the BTTS to a marker on the surface of a target cell activates expression of the anti-inflammatory payload by the cell. The payload can be an anti-inflammatory cytokine, an antibody that inhibits pro-inflammatory signaling, a soluble receptor that binds to pro-inflammatory cytokine, a cytokine sink or any combination thereof, for example. In any embodiment, the cell may express CD25, which not only acts as a cytokine sink but it also causes the engineered CD4+ T cell to proliferate when it binds to its ligand. Stimulation of CD25 should increase survival/persistence of the T cells in the host.

In any embodiment, the BTTS may comprise: i. an extracellular binding domain that binds to a CNS-specific cell surface marker (e.g., MOG, CDH10, CSPG5, PTPRZ1, BCAN, GRM3, Neurexin 1b or NrCAM), ii. a transmembrane domain; and ii. an intracellular domain comprising a transcriptional activator, where binding of the extracellular binding domain to the cell surface marker on the surface of another cell induces proteolytic cleavage BTTS to release the transcriptional activator. In these embodiments, the released transcriptional activator induces expression of the payload by the cell. In any embodiment, the payload may be a natural molecule. In other embodiments, the payload may be an engineered (i.e., non-natural molecule).

In any embodiment, the immunosuppressive cell may be a T cell, a B cell, a macrophage, or a neutrophil. For example, in some embodiments, the immunosuppressive immune cell may be a CD4+ T cell.

In any embodiment, the BTTS may activated by binding to BCAN and the anti-inflammatory payload comprises IL-10, the BTTS may be activated by binding to CDH10 and the anti-inflammatory payload comprises IL-10, the BTTS may be activated by binding to BCAN and the anti-inflammatory payload comprises TGFβ or the BTTS is activated by binding to CDH10 and the anti-inflammatory payload comprises TGFβ. In these embodiments, the BTTS may be activated by binding to BCAN or CDH10 and the anti-inflammatory payload May further comprise CD25 and/or IL-2 or a variant thereof.

### Binding-Triggered Transcriptional Switches (BTTSs)

The BTTS is a cleavable fusion protein that contains: (a) an extracellular binding domain comprising a protein binding domain (e.g., scFv or nanobody) that binds to brain-specific cell surface marker (e.g., MOG, CDH10, CSPG5, PTPRZ1, BCAN, GRM3, Neurexin 1b or NrCAM) on a cell, (b) a transmembrane domain, and (c) an intracellular domain comprising a transcriptional activator, where binding of the binding domain to the marker on the surface of the other cell induces proteolytic cleavage of the BTTS to release the transcriptional activator. In some embodiments, the BTTs may additionally contain an extracellular force sensing region between regions (a) and (b) and (d) one or more force-dependent cleavage sites in (c) that are cleaved when the force sensing region is activated.

In this switch, the fusion protein is cleaved to release the intracellular domain when the extracellular domain of the fusion protein engages with a marker on another cell. As such, in many cases, the fusion protein will contain a force sensing region (which is typically in the extracellular domain) and one or more force-dependent cleavage sites that are cleaved when the force sensing region is activated. The position of the force-dependent cleavage sites may vary and, in some embodiments the fusion protein may contain at least two cleavage sites. In some cases, one of the cleavage sites may be extracellular and the other may be in the transmembrane domain or within 10 amino acids of the transmembrane domain in the intracellular domain. In any embodiment, the force sensing region and/or the one or more force-dependent cleavage sites may be from a Delta/Serrate/Lag2 (DSL) superfamily protein, as reviewed by Pintar et al (Biology Direct 2007 2:1-13). For example, the force sensing region and/or the one or more force-dependent cleavage sites may be from Notch (see Morsut Cell. 2016 164:780-91), von Willebrand Factor (vWF), amyloid-beta, CD16, CD44, Delta, a cadherin, an ephrin-type receptor or ephrin ligand, a protocadherin, a filamin, a synthetic E cadherin, interleukin-1 receptor type 2 (IL1R2), major prion protein (PrP), a neuregulin or an adhesion-GPCR. Several other examples of this type of protein are known and listed in Pintar, supra. Many members of this family appear to share a similar architecture a region that unfolds and opens up a protease cleavage site (e.g., EGF-like repeats; see Cordle et al Nat. Struct. Mol. Biol. 2008 15:849-857), a trans-membrane segment, and a relatively short (˜100-150 amino acids) intracellular domain. These sequences permit the binding-triggered release of a transcriptional activator from the membrane in their natural environment and can be readily adapted herein.

In some cases, the one or more ligand-inducible proteolytic cleavage sites are selected from S1, S2, and S3 proteolytic cleavage sites. In some cases, the S1 proteolytic cleavage site is a furin-like protease cleavage site comprising the amino acid sequence Arg-X-(Arg/Lys)-Arg, where X is any amino acid. In some cases, the S2 proteolytic cleavage site ADAM-17-type protease cleavage site comprising an Ala-Val dipeptide sequence. In some cases, the S3 proteolytic cleavage site is a γ-secretase cleavage site comprising a Gly-Val dipeptide sequence. The S3 proteolytic cleavage site is in the transmembrane domain. In many cases, the shear force generated by binding of the extracellular domain of this fusion protein to another cells unfolds the force sensing region (which, in the case of Notch contains EGF-like repeats whereas in other protein is made up of other sequences such as the A2 domain in vWF (see, e.g., J Thromb Haemost. 2009 7:2096-105, Lippok Biophys J. 2016 110:545-54, Lynch Blood. 2014 123: 2585-92, Crawley, Blood. 2011 118:3212-21 and Xy J Biol Chem. 2013 288:6317-24) or modified A2 domain that has, e.g., the R1597W, E1638K and I1628T substitutions. The architecture of such proteins is described in, e.g., Morsut et al, Cell. 2016 164:780-91, WO2016138034 and WO2019099689, among other places).

In some cases, the fusion protein includes an S1 ligand-inducible proteolytic cleavage site. An S1 ligand-inducible proteolytic cleavage site can be located between the HD-N segment and the HD-C segment. In some cases, the S1 ligand-inducible proteolytic cleavage site is a furin-like protease cleavage site. A furin-like protease cleavage site can have the canonical sequence Arg-X-(Arg/Lys)-Arg, where X is any amino acid; the protease cleaves immediately C-terminal to the canonical sequence. For example, in some cases, an amino acid sequence comprising an S1 ligand-inducible proteolytic cleavage site can have the amino acid sequence GRRRRELDPM (SEQ ID NO:1), where cleavage occurs between the “RE” sequence. As another example, an amino acid sequence comprising an S1 ligand-inducible proteolytic cleavage site can have the amino acid sequence RQRRELDPM (SEQ ID NO:2), where cleavage occurs between the “RE” sequence.

In some cases, the fusion protein polypeptide includes an S2 ligand-inducible proteolytic cleavage site. An S2 ligand-inducible proteolytic cleavage site can be located within the HD-C segment. In some cases, the S2 ligand-inducible proteolytic cleavage site is an ADAM-17-type protease cleavage site. An ADAM-17-type protease cleavage site can comprise an Ala-Val dipeptide sequence, where the enzyme cleaves between the Ala and the Val. For example, in some cases, amino acid sequence comprising an S2 ligand-inducible proteolytic cleavage site can have the amino acid sequence KIEAVKSE (SEQ ID NO:3), where cleavage occurs between the “AV” sequence. As another example, an amino acid sequence comprising an S2 ligand-inducible proteolytic cleavage site can have the amino acid sequence KIEAVQSE (SEQ ID NO: 4), where cleavage occurs between the “AV” sequence.

In some cases, the fusion protein includes an S3 ligand-inducible proteolytic cleavage site. An S3 ligand-inducible proteolytic cleavage site can be located within the TM domain. In some cases, the S3 ligand-inducible proteolytic cleavage site is a gamma-secretase (γ-secretase) cleavage site. A γ-secretase cleavage site can comprise a Gly-Val dipeptide sequence, where the enzyme cleaves between the Gly and the Val. For example, in some cases, an S3 ligand-inducible proteolytic cleavage site has the amino acid sequence VGCGVLLS (SEQ ID NO:5), where cleavage occurs between the “GV” sequence. In some cases, an S3 ligand-inducible proteolytic cleavage site comprises the amino acid sequence GCGVLLS (SEQ ID NO:6).

In some cases, the fusion protein polypeptide lacks an S1 ligand-inducible proteolytic cleavage site. In some cases, the BTTS lacks an S2 ligand-inducible proteolytic cleavage site. In some cases, the BTTS lacks an S3 ligand-inducible proteolytic cleavage site. In some cases, the BTTS lacks both an S1 ligand-inducible proteolytic cleavage site and an S2 ligand-inducible proteolytic cleavage site. In some cases, the BTTS includes an S3 ligand-inducible proteolytic cleavage site; and lacks both an S1 ligand-inducible proteolytic cleavage site and an S2 ligand-inducible proteolytic cleavage site.

In some embodiments, the fusion protein may have an vWF A2 sequence or a variation thereof, an ADAMTS13 cleavage site (which may be described by the consensus sequence HEXXHXXGXXHD (SEQ ID NO:7); Crawley, Blood. 2011 118:3212-21), and an S3 or γ-secretase cleavage site, although many other arrangements exist. In some embodiments, the switch may contain components that are borrowed from Notch. In other embodiments, the switch may not contain components that are from Notch.

For simplicity, BTTSs, including but not limited to chimeric notch receptor polypeptides, are primarily single polypeptide chains. However, BTTSs, including chimeric notch receptor polypeptides, may be divided or split across two or more separate polypeptide chains where the joining of the two or more polypeptide chains to form a functional BTTS, e.g., a chimeric notch receptor polypeptide, may be constitutive or conditionally controlled. For example, constitutive joining of two portions of a split BTTS may be achieved by inserting a constitutive heterodimerization domain between the first and second portions of the split polypeptide such that upon heterodimerization the split portions are functionally joined.

Useful BTTSs that may be employed in the subject methods include, but are not limited to, modular extracellular sensor architecture (MESA) polypeptides. A MESA polypeptide comprises: a) a ligand binding domain; b) a transmembrane domain; c) a protease cleavage site; and d) a functional domain. The functional domain can be a transcription regulator (e.g., a transcription activator, a transcription repressor). In some cases, a MESA receptor comprises two polypeptide chains. In some cases, a MESA receptor comprises a single polypeptide chain. Non-limiting examples of MESA polypeptides are described in, e.g., U.S. Patent Publication No. 2014/0234851; the disclosure of which is incorporated herein by reference in its entirety.

Useful BTTSs that may be employed in the subject methods include, but are not limited to, polypeptides employed in the TANGO assay. The subject TANGO assay employs a TANGO polypeptide that is a heterodimer in which a first polypeptide comprises a tobacco etch virus (Tev) protease and a second polypeptide comprises a Tev proteolytic cleavage site (PCS) fused to a transcription factor. When the two polypeptides are in proximity to one another, which proximity is mediated by a native protein-protein interaction, Tev cleaves the PCS to release the transcription factor. Non-limiting examples of TANGO polypeptides are described in, e.g., Barnea et al. (Proc Natl Acad Sci USA. 2008 Jan. 8; 105 (1): 64-9); the disclosure of which is incorporated herein by reference in its entirety.

Useful BTTSs that may be employed in the subject methods include, but are not limited to von Willebrand Factor (vWF) cleavage domain-based BTTSs, such as but not limited to e.g., those containing a unmodified or modified vWF A2 domain. A subject vWF cleavage domain-based BTTS will generally include: an extracellular domain comprising a first member of a binding pair; a von Willebrand Factor (vWF) cleavage domain comprising a proteolytic cleavage site; a cleavable transmembrane domain and an intracellular domain. Non-limiting examples of vWF cleavage domains and vWF cleavage domain-based BTTSs are described in Langridge & Struhl (Cell (2017) 171 (6): 1383-1396); the disclosure of which is incorporated herein by reference in its entirety.

Useful BTTSs that may be employed in the subject methods include, but are not limited to chimeric Notch receptor polypeptides, such as but not limited to e.g., synNotch polypeptides, non-limiting examples of which are described in PCT Pub. No. WO 2016/138034, U.S. Pat. Nos. 9,670,281, 9,834,608, Roybal et al. Cell (2016) 167 (2): 419-432, Roybal et al. Cell (2016) 164 (4): 770-9, and Morsut et al. Cell (2016) 164 (4): 780-91; the disclosures of which are incorporated herein by reference in their entirety. The “SNIPR” switch is another example of a BTTS (see Zhu et al 2022 Cell. 185:1431-1443 and WO2021061856), although others exist and/or can be readily designed.

Expression of the BTTS in the cell may be constitutive or inducible, e.g., by binding of another BTTS to an antigen on another cell in the patient.

Examples of transcriptional activators that can be part of the fusion protein are numerous and include artificial transcription factors (ATFs) such as, e.g., Zinc-finger-based artificial transcription factors (including e.g., those described in Sera T. Adv Drug Deliv Rev. 2009 61 (7-8): 513-26; Collins et al. Curr Opin Biotechnol. 2003 14 (4): 371-8; Onori et al. BMC Mol Biol. 2013 14:3. In some cases, the transcriptional activator may contain a GAL4 DNA binding domain, which binds to the Gal4 responsive UAS, which has been well characterized in the art. Examples of suitable transcriptional activators include GAL4-VP16 and GAL4-VP64, although many others could be used. As would be appreciated, the identity of the transcription activators may vary. In some embodiments, the transcription factor may have a DNA binding domain that binds to a corresponding promoter sequence and an activation domain. In many embodiments, the DNA binding domain transcription factor may be independently selected from Gal4-, LexA-, Tet-, Lac-, dCas9-, zinc-finger- and TALE-based transcription factors. TALE- and CRISPR/dCas9-based transcription factors are described in Lebar (Methods Mol Biol. 2018 1772:191-203), among others. The binding sites for such domains are well known or can be designed at will. The transcription factors can have any suitable activation domain, e.g., VP16, VP64, Ela, Sp1, VP16, CTF, GAL4 among many others.

### Extracellular Binding Domains

The extracellular binding domain of the BTTS may bind to a CNS-specific cell-surface marker such as MOG, CDH10, CSPG5, PTPRZ1, BCAN, GRM3, Neurexin 1b or NrCAM. The binding domains can be a single chain Fv or nanobody although, in theory, one could also use a cognate receptor or ligand for the binding domain instead. In these embodiments, the term “single-chain Fv” or “scFv” antibody fragments comprise the VH and VL domains of antibody, wherein these domains are present in a single polypeptide chain. In some embodiments, the scFv polypeptide further comprises a polypeptide linker between the VH and VL domains, which enables the scFv to form the desired structure for antigen binding. For a review of scFv, see Pluckthun in The Pharmacology of Monoclonal Antibodies, vol. 113, Rosenburg and Moore eds., Springer-Verlag, New York, pp. 269-315 (1994). The term “nanobody” (Nb), as used herein, refers to the smallest antigen binding fragment or single variable domain (VHH) derived from naturally occurring heavy chain antibody and is known to the person skilled in the art. They are derived from heavy chain only antibodies, seen in camelids (Hamers-Casterman et al. (1993) Nature 363:446; Desmyter et al. (2015) Curr. Opin. Struct. Biol. 32:1). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody.

### Anti-Inflammatory Cytokines

If the protein induced by the BTTS binding to its target is an anti-inflammatory cytokine, the cytokine will be secreted from the cell. In these embodiments, the circuit may comprise a nucleic acid containing a promoter that is activated by the released transcriptional activator, and a coding sequence encoding an anti-inflammatory cytokine. In this disclosure, the term “anti-inflammatory cytokine” is intended to encompass natural molecules that have anti-inflammatory activity (e.g., Il-1ra, IL-4, IL-10, IL-11, IL-13, IL-35 and TGF-β), as well as non-natural or “engineered” cytokines that have anti-inflammatory activity. As would be appreciated, cytokines arc secreted from the cell and their coding sequence will encode a secretion signal. Anti-inflammatory cytokines of particular interest include IL-10, TGF-β, IL-4, and IFNβ. Anti-inflammatory cytokines of particular interest include IL-10, TGF-b, IL-4, and IFNb. IL10 variants are described in Saxton et al (Science 2021 371:6535); TGFb mimics are described in Johnston et al (Science Immun. 2020 5:50); IL35 variants are described in Collison et al (Science 2021 371:6535); and CD25-biased IL2 variants are described in Khoryati et al (Science Imm. 2020 5:50). IL2 and its variants can also be used, since this cytokine can recruit Tregs, which have indirect anti-inflammatory properties.

### Pro-Inflammatory Cytokine Sinks

The term “pro-inflammatory cytokine sink” is intended to refer to a protein that specifically binds to a pro-inflammatory cytokine (e.g., IL-2, CCL-21, IL-12, IL-7, IL-15 or IL-21, etc.) and prevents it from binding with its cognate receptor on another immune cell. In some embodiments, the cytokine sink comprises at least the extracellular domain of a receptor for a pro-inflammatory cytokine, e.g., at least the extracellular domain of IL-1R, IL-2R/CD25, IL-12R, IL-18R, TNFR1, TNFR2, IFNGR, GM-CSFR, etc., or a part thereof that binds to its cognate ligand. For example, the cytokine sink may have the extracellular domain of IL-1R (which binds to IL-1), IL-2R or CD25 (which binds to IL-2), IL-12R, IL-18R (which binds to IL-18), TNFR1 and TNFR2 (which binds to TNF-α), IFNGR (which binds to IFNγ) and GM-CSFR (which binds to GMCSF) or a subunit thereof that binds to its ligand. This domain may be tethered to the cell via a transmembrane domain or it may be secreted. If the domain is tethered to the cell then, in some embodiments, a truncated or mutated form of the receptor may be used so that the receptor is incapable of signaling. In other embodiments, the full-length receptor may be used. In these embodiments, the cell may not have the internal machinery to transduce a signal from that receptor to the nucleus. In one embodiment, a sink may contain the extracellular domain of CD25 (which is the receptor for IL-2), although others could be used too. Anti-IL17 antibodies can also be used.

In alternative embodiments, an antibody (e.g., a scFv) that binds to the pro-inflammatory cytokine may be used. In these embodiments, the antibody may be tethered to the cell, e.g., via a transmembrane domain, or secreted.

In any embodiment, the payload may be an inhibitor selected from an anti-IL-6 antibody, soluble TNFR (sTNFR), and an anti-CD20 antibody.

In these embodiments, the circuit may comprise a nucleic acid containing a promoter that is activated by the released transcriptional activator, and a coding sequence encoding a pro-inflammatory cytokine sink.

### Ectonucleotidase

Ectonucleotidases are nucleotide metabolizing enzymes that are expressed on the plasma membrane and have externally oriented active sites. These enzymes metabolize nucleotides to nucleosides. Extracellular adenosine generated by the ectonucleotidases CD39 and CD73 is a newly recognized “immune checkpoint mediator” that is believed to interfere with anti-tumor immune responses. Expressing an ectonucleotidase such as CD39 or CD73 on a cell should dampen the immune response around that cell. In these embodiments, the circuit may comprise a nucleic acid containing a promoter that is activated by the released transcriptional activator, and a coding sequence encoding a ectonucleotidase.

### Neurotrophic Factors

In any embodiment, the payload may be a neurotrophic factor selected from BDNF, NGF and NT-3.

### Combinations

As noted above, in some embodiments expression of two or more payloads may be induced by binding of the BTTS to the cell surface marker. In these embodiments, the different proteins may be on different constructs with the same promoter or their expression may be coordinated by an IRES. Other ways for co-expressing two proteins are known. The two or more payloads may be on the same vector or different vectors.

### Cells

Immune cells encoding a circuit of the present disclosure may be generated by any convenient method. Nucleic acids encoding one or more components of a subject circuit may be stably or transiently introduced into the subject immune cell, including where the subject nucleic acids are present only temporarily, maintained extrachromosomally, or integrated into the host genome. Introduction of the subject nucleic acids and/or genetic modification of the subject immune cell can be carried out in vivo, in vitro, or ex vivo.

In some cases, the introduction of the subject nucleic acids and/or genetic modification is carried out ex vivo. For example, a primary cell is obtained from an individual; and the cell obtained from the individual is modified to express components of a circuit of the present disclosure. In other embodiments, an non-immunogenic allogeneic cell may be used.

### Circuits

As noted above, binding of a BTTS to the cell surface marker on another cell activates expression of one or more other proteins. In these embodiments, binding of the binding domain of the BTTS to the antigen on the surface of a cell induces proteolytic cleavage of the BTTS to release the transcriptional activator. The released transcriptional activator then binds to a promoter that drives the expression of the one or more other proteins, thereby inducing expression of the one or more other proteins. The general principles of a circuit are described in WO 2016/138034, U.S. Pat. Nos. 9,670,281, 9,834,608, Roybal et al. Cell (2016) 167 (2): 419-432, Roybal et al. Cell (2016) 164 (4): 770-9, and Morsut et al. Cell (2016) 164 (4): 780-91, among others.

### Methods of Treatment

A method of treatment is described below. In general terms, this method may comprise administering a cell described above to the subject in vivo. In some embodiments, primary immune cells may be purified from an individual, constructs encoding the above proteins may be introduced into the cells ex vivo, and the recombinant cells may be expanded and administered to the subject, e.g., by injection. In other embodiments, pre-made allogeneic cells (which may have abrogated MHC class I molecules) may be used instead.

In some embodiments, the subject may have an inflammatory brain disease, e.g., multiple sclerosis, neuromyelitis optica (NMO), anti-myelin oligodendrocyte glycoprotein antibody disorder (MOG), autoimmune encephalitis, transverse myelitis, optic neuritis, neurosarcoidosis, neuro-Behcet's disease, a MOG antibody-associated diseases, paraneoplastic diseases affecting the CNS, neuropsychiatric systemic lupus erythematosus (NP-SLE) or Aicardi-Goutieres syndrome (AGS), for example.

T cells may be obtained from any suitable source. For example, T cells may be differentiated in vitro from a hematopoietic stem cell population, or T cells may be obtained from a subject. T cells may be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by reference in its entirety. In any embodiment, the cells that are expanded can be a primary T cell.

The therapeutic cells may be autologous/autogeneic (“self”) or non-autologous (“non-self,” e.g., allogeneic, syngeneic or xenogeneic). “Autologous” as used herein, refers to cells obtained from the subject to whom the therapeutic cells are later administered. “Allogeneic” as used herein refers to cells obtained from a donor other than the subject to whom the therapeutic cells are administered. In some embodiments, the cells (e.g., T cells) are cells obtained from a mammalian subject. In certain embodiments, the mammalian subject is a primate. In some embodiments, the cells are obtained from a human.

By treatment is meant at least an amelioration of one or more symptoms associated with the condition of the subject, where amelioration is used in a broad sense to refer to at least a reduction in the magnitude of a parameter, e.g., symptom, associated with the condition being treated. As such, treatment also includes situations where the condition, or at least one or more symptoms associated therewith, are completely inhibited, e.g., prevented from happening, or stopped, e.g., terminated, such that the subject no longer suffers from the condition, or at least the symptoms that characterize the condition.

In some embodiments, a combination of cells may be used. For example, in some embodiments different cells that recognize the same CNS marker (e.g., MOG, CDH10 or BCAN) but express a different payloads (e.g., TGFβ and IL-10) may be used in combination.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular (ly); i.p., intraperitoneal (ly); s.c., subcutaneous (ly); and the like.

### Materials and Methods

Construct Design: SynNotch receptors were built by fusing the various scFv sequences (Sidhu lab) to mouse Notch1 (NM_008714) minimal regulatory region (res.1427-1752) and Gal4 DBD VP64. All synNotch receptors contain N-terminal CD8a signal peptide (MALPVTALLLPLALLL HAARP; SEQ ID NO:8) for membrane targeting and α-myc-tag (EQKLISEEDL; SEQ ID NO:9) for detecting surface expression with α-myc A647 (cell-signaling #2233); see Morsut et al. (4) for synNotch sequence. Receptors were cloned into a modified pHR′SIN: CSW vector containing a PGK or SFFV promoter. The pHR′SIN: CSW vector was also used to make response element plasmids with five copies of the Gal4 DNA binding domain target sequence (GGAGCACTGTCCTCCGAACG; SEQ ID NO:10) upstream from a minimal CMV promoter. Response element plasmids also contain a PGK promoter that constitutively drives blue fluorescent protein (BFP) expression to easily identify transduced T cells. CARs were built by fusing the binding scFv of the targeted antigen to the hinge region of the human CD8a chain and transmembrane and cytoplasmic regions of the human 4-1BB, and CD3z signaling domains. Inducible constructs were cloned into a site 3′ to the Gal4 response elements. CARs were tagged c-terminally with GFP or RFP, or n-terminally with myc tag, or flag tag to verify surface expression.

Primary Human T Cell Isolation and Culture: Primary CD4⁺ and CD8⁺ T cells were isolated from donor blood after apheresis by negative selection (STEMCELL Technologies #15062 and #15063). Blood was obtained from Blood Centers of the Pacific, as approved by the University of California San Francisco (UCSF) Institutional Review Board. T cells were cryopreserved in RPMI-1640 (UCSF cell culture core) with 20% human AB serum (Valley Biomedical, #HP1022) and 10% dimethyl sulfoxide. After thawing, T cells were cultured in human T cell medium consisting of X-VIVO 15 (Lonza #04-418Q), 5% Human AB serum, and mM neutralized N-acetyl L-Cysteine (Sigma-Aldrich #A9165) supplemented with 30 units/mL interleukin (IL)-2 (NCI BRB Preclinical Repository);

Lentiviral Transduction of Human T Cells: Pantropic vesicular stomatitis virus G (VSV-G) pseudotyped lentivirus was produced via transfection of Lenti-X 293T cells (Clontech #11131D) with a pHR′SIN: CSW transgene expression vector and the viral packaging plasmids pCMVdR8.91 and pMD2.G using Fugene HD (Promega #E2312). Primary T cells were thawed the same day and, after 24 hours in culture, were stimulated with Human T-Activator CD3/CD28 Dynabeads (Life Technologies #11131D) at a 1:3 cell: bead ratio. At 48 hours, viral supernatant was harvested and, in some assays, concentrated using Lenti-X concentrator (Clontech #631231). Primary T cells were exposed to the lentivirus for 24 hours. At day 5 after T cell stimulation, Dynabeads were removed, and sorted with a Beckton Dickinson (BD) FACS ARIA Fusion or Sony SH800S Cell Sorter. AND-gate T cells exhibiting basal CAR expression were gated out during sorting. T cells were expanded until rested and could be used in assays.

Human T Cell Phenotyping: T cells phenotypes were assessed using the following antibodies: PE anti-CD25 (clone BC96, 302606, BioLegend) for T cell activation; APC-Cy7 anti-IL10.

Cell Lines: Cell lines used were K562 myelogenous leukemia cells (ATCC #CCL-243),

In Vitro Stimulation of SynNotch T cells: For in vitro synNotch induction, the engineered T cell and K562 cells were cultured in a 96-well plate for 48 h. When cytokine release assays were conducted, the supernatant was collected and processed by ELISA. Assessment of autoreactive T cell activation: CD4⁺ control or synNotch-IL10 T cells were cultured with K562s expressing BCAN, CD4⁺, CD25⁺ MOG TCR (isolated from C57BL/6-Tg (Tcra2D2,Tcrb2D2) 1Kuch/J mice, Jackson lab), APC (splenic cells that are CD4, isolated from C57BL/6-Tg (Tcra2D2,Tcrb2D2)1Kuch/J mice, Jackson lab) in presence of MOG P35-55 (50 μg/mL) at 1:1:1:1 ratio (5×10⁴ each) for 4 days. Cells and supernatant were further processed as described in figures.

Assessment of microglial inflammation inhibition: CD4⁺ control or synNotch-IL10 T cells were cultured with K562s expressing BCAN, BV2 microglia cells, 1×10⁵, 1×10⁵ and 1×10⁴ respectively for 24 hours in media containing LPS (100 ng/ml) and murine IFN-γ (0.5 ng/mL). Cells and supernatant were further processed as described in above.

In Vivo Mouse Experiments: All mouse experiments were conducted according to Institutional Animal Care and Use Committee (IACUC)-approved protocols.

In the EAE experiments, an adoptive transfer model was used as previously described. Briefly, naive 8-14 week-old C57BL/6 female mice were injected subcutaneously with 50 μg/mouse of MOG peptide in 0.1 ml emulsion of CFA containing 4 mg/ml Mycobacterium tuberculosis H37Ra (DIFCO Laboratories) and PBS (1:1). Ten days later, draining lymph nodes were collected, single-cell suspensions were prepared and cells were stimulated at 5×10⁶ cells/ml with 25 μg/ml of relevant MOG peptide in the presence of recombinant murine IL-12 (25 ng/ml) and anti-murine IFN-γ mAb (BD). After three days of culture, cells were harvested, washed, and 20-25×10⁶ cells were injected i.p. into each in naive recipient RAG1^{-/-} mice.

### Example 1

### synNotch Induced Production of Suppressive Cytokine TGFb

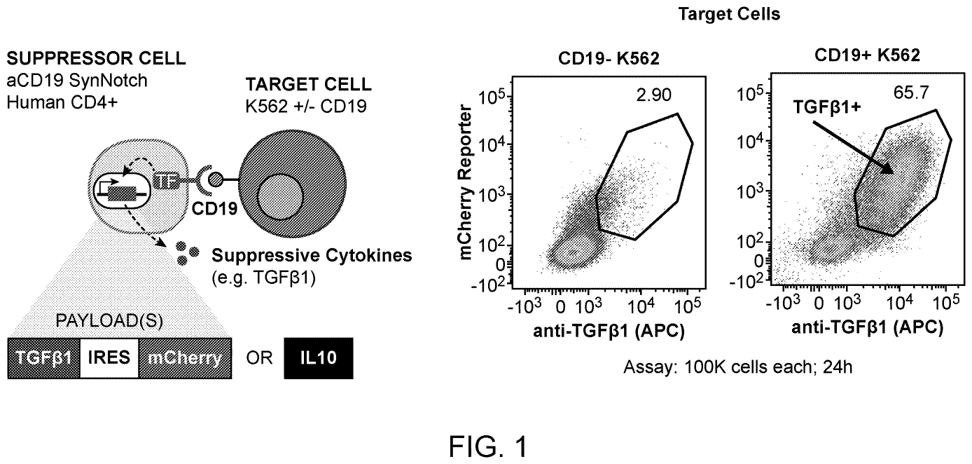

Engineered immune cells can produce immuno-suppressive payloads in response to a specific antigen. Human CD4+ T cells can selectively induce immune inhibitory cytokine TGFβ1 in response to CD19 antigen using SynNotch (measured by flow cytometry). The results shown in FIG. 1 show that expression of the suppressive cytokine TGFb can be induced by synNotch binding to CD19 on another cell.

### Example 2

### Suppressor Cells that Produce Combination of TGFb (Suppressive Cytokine) and CD25 (IL2 Sink) are Very Effective at Suppressing CAR T Killing In Vitro

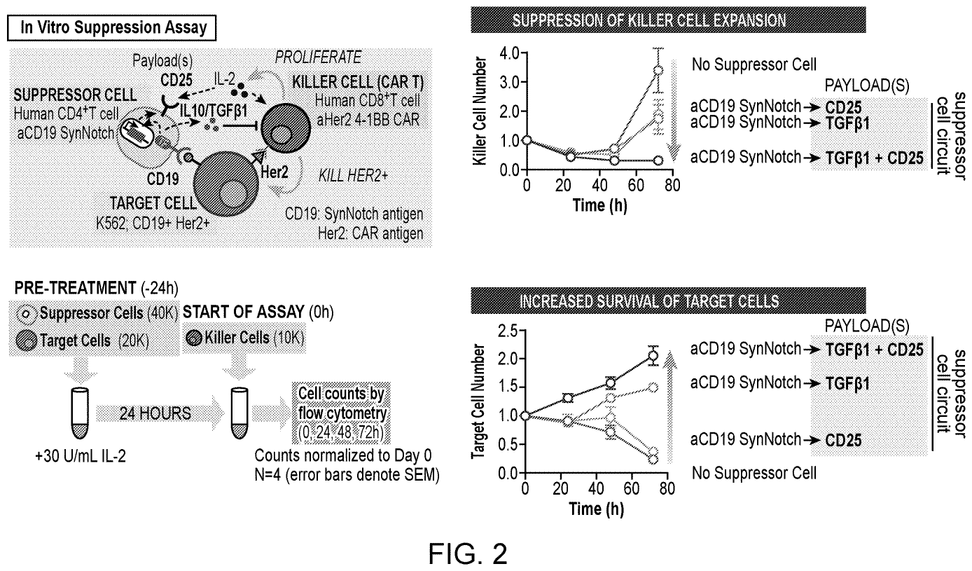

T cells inducibly producing a combination of inhibitory cytokine TGFβ1 and pro-inflammatory cytokine sink CD25 using synNotch show strong suppression of CAR T cell proliferation and killing in vitro. In vitro immune suppression was assayed by co-culturing three cells: (1) human CD4+ T cells with anti-CD19 SynNotch inducing production of CD25, TGFβ1, or both payloads, (2) K562 target cells expressing both a synNotch antigen, CD19, and a CAR antigen, Her2, and (3) human CD8+ T cells expressing an anti-Her2 4-1BB CAR. Cell counts were tracked over time using flow cytometry. Human CD4+ T cells with synNotch inducing production of both payloads is most effective at suppressing CAR T cell activity, proliferation of the CAR T cells and killing of the K562 target cells. See FIG. 2.

### Example 3

### Suppressor Cells that Produce Combination of IL10 (Suppressive Cytokine) and CD25 (IL2 Sink) are Very Effective at Suppressing CAR T Killing In Vitro

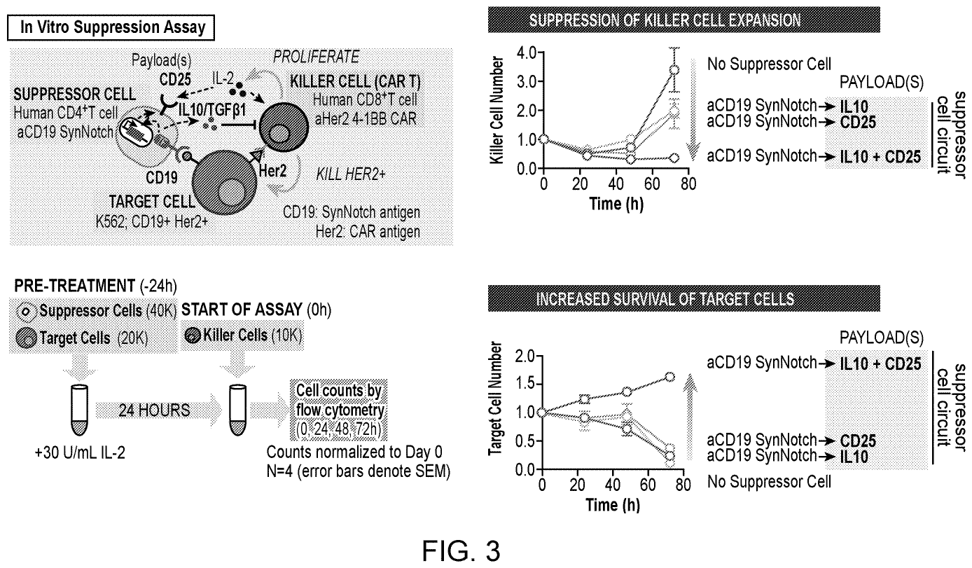

T cells inducibly producing a combination of inhibitory cytokine IL10 and pro-inflammatory cytokine sink CD25 using synNotch show strong suppression of CAR T cell proliferation and killing in vitro. In vitro immune suppression was assayed by co-culturing three cells: (1) human CD4+ T cells with anti-CD19 SynNotch inducing production of CD25, IL10, or both payloads, (2) K562 target cells expressing both a synNotch antigen, CD19, and a CAR antigen, Her2, and (3) human CD8+ T cells expressing an anti-Her2 4-1BB CAR. Cell counts were tracked over time using flow cytometry. Human CD4+ T cells with synNotch inducing production of both payloads is most effective at suppressing CAR T cell activity, proliferation of the CAR T cells and killing of the K562 target cells. See FIG. 3.

### Example 4

### Suppressor Cells that Produce Combination of TGFb (Suppressive Cytokine) and CD25 (IL2 Sink) are Very Effective at Suppressing CAR T Killing of Tumors In Vivo

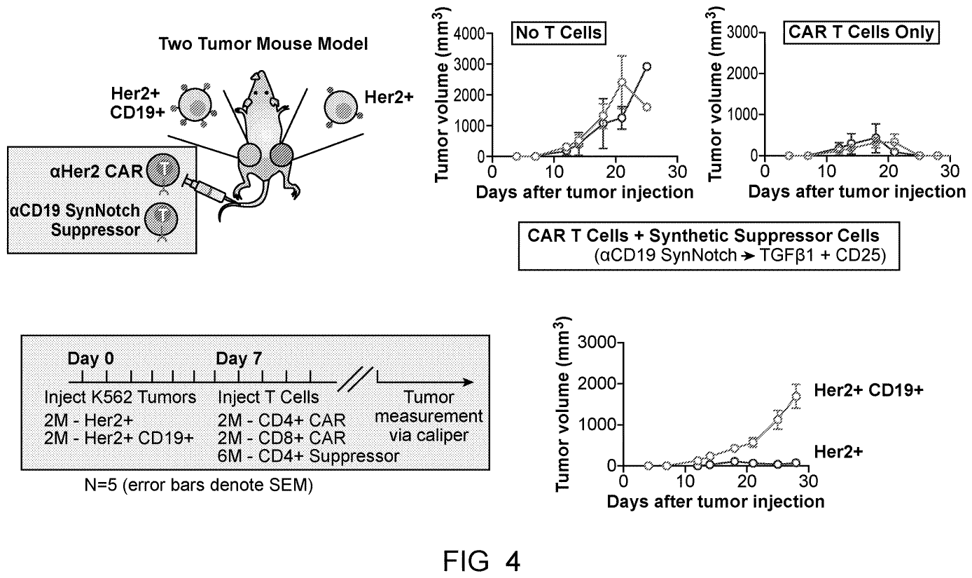

Synthetic immune-suppressive cells can locally suppress immune response. K562 tumors, Her2+ and Her2+CD19+, were subcutaneously injected in the flanks of N.S.G. mice. These mice were treated with either no T cells, anti-Her2 CAR T cells only, or anti-Her2 CAR T cells and synthetic suppressor cells (human CD4+ T cells with an anti-CD19 synNotch induciblely producing TGFβ1 and CD25) by i.v. injection after 7 days. Tumor volume was monitored by caliper measurement. Synthetic suppressor cells show strong local suppression of CAR T cell killing in the CD19+ tumor without suppressing CAR T cell killing of the CD19-tumor. See FIG. 4.

### Example 5

### Engineered T Cells Overexpressing CD25 Increases Consumption of IL2 and Cell Proliferation

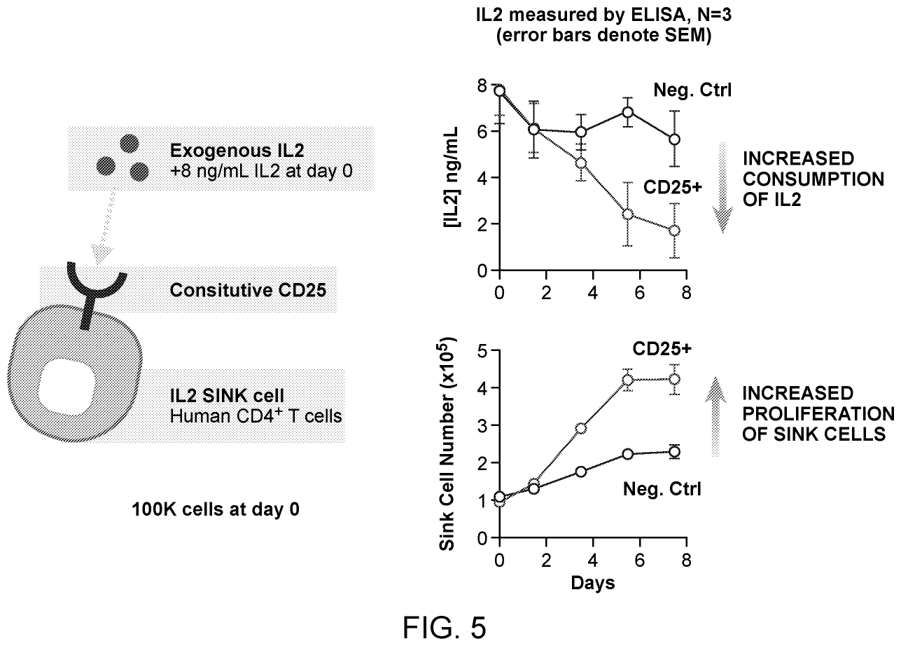

Human CD4+ T cells constitutively expressing CD25 show increased consumption of IL2 (measured by ELISA) and increased proliferation (measured by flow cytometry) in vitro when exogenous IL2 is added to the media. Cells that express CD25 cell should also have a survival advantage. See FIG. 5.

### Example 6

### synNotch→IL10 Synthetic Suppressor Cells can Block Autoimmune Cell Proliferation in Brain and CNS in Mouse Neuroinflammation Model

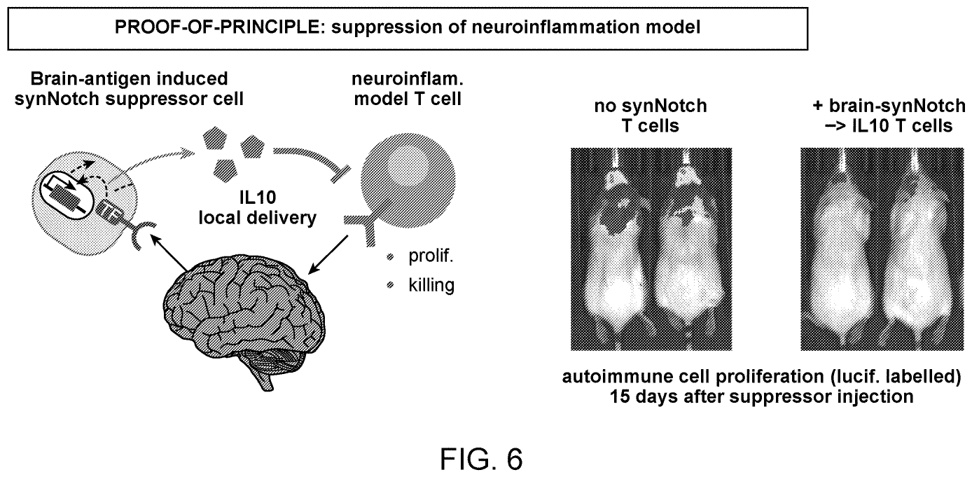

IL10 expression can be activated by mouse brain specific antigen (CDH10). See FIG. 6.

### Example 7

### Engineered Primary CD4+ T Cells can Decrease Brain Inflammation in a Model System

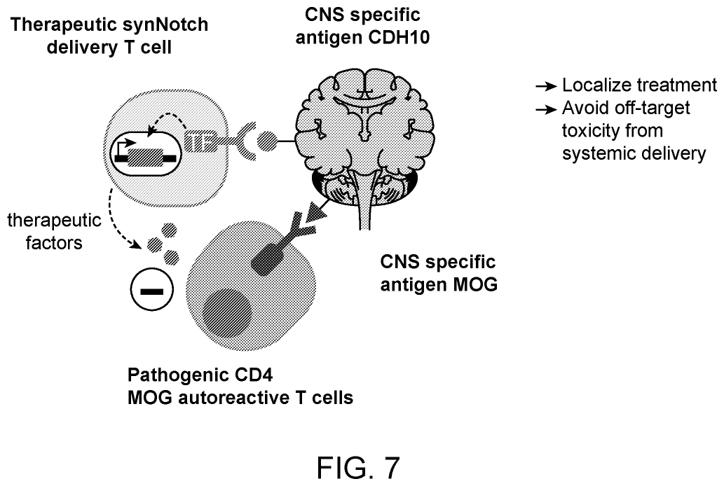

FIG. 7 illustrates a strategy for inhibiting pathogenic autoreactive T cells to reduce their pathogenicity. These CNS-targeted therapeutic cells were tested in an adoptive transfer model that uses encephalitogenic MOG autoreactive T cells.

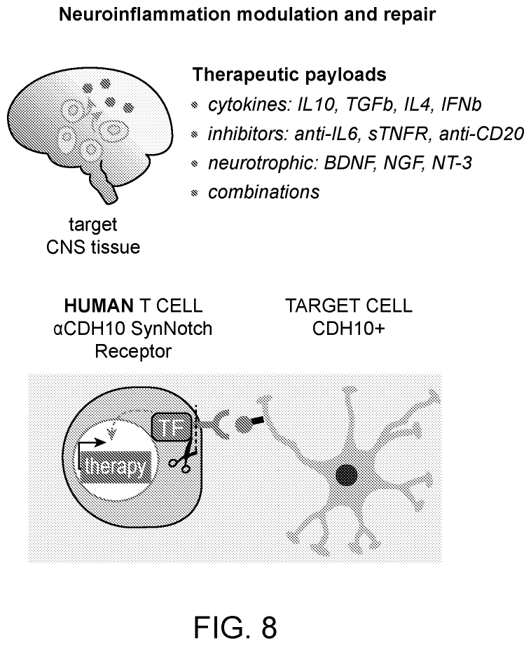

FIG. 8 primary human T cells were engineered to express therapeutic payloads such as the ones described in panel A, using a CNS-specific synNotch. These cells work as expected as shown by intracellular stain and by ELISA (see a later figure). This figure shows that primary human CD4 T cells can be engineered to secrete therapeutic payloads upon recognition of a CNS-specific antigen.

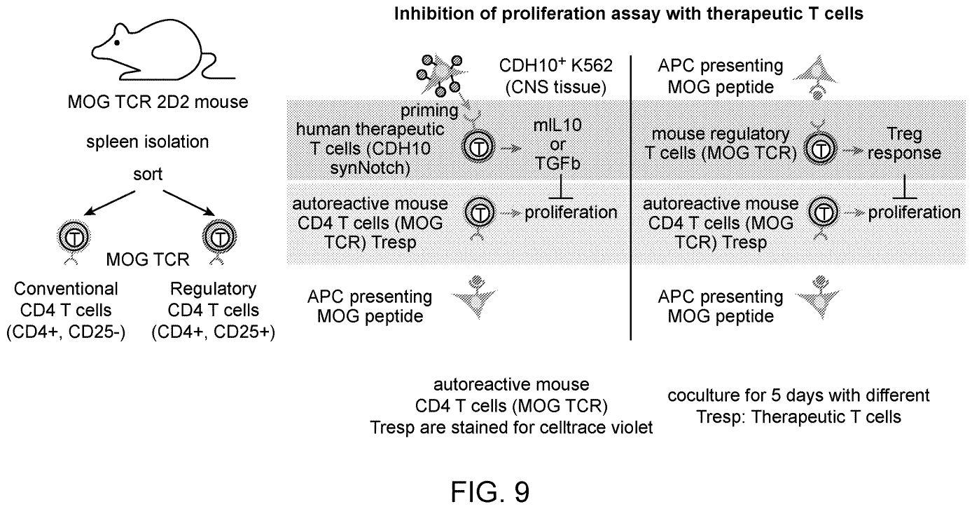

FIG. 9 generally describes the in vitro assay used to determine if engineered human T cells can inhibit the proliferation of MOG autoreactive mouse T cells.

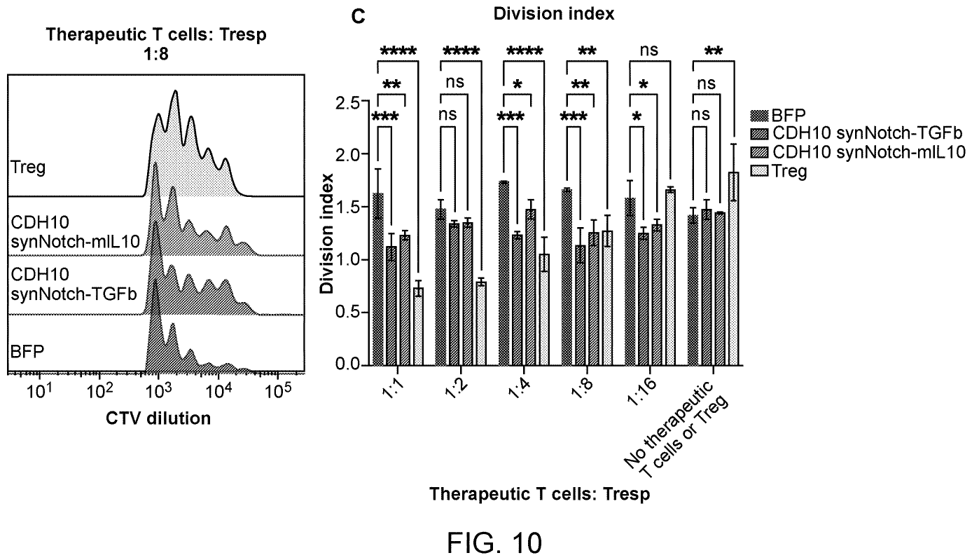

FIG. 10 cells containing a CDH10 synNotch construct were tested for inhibition of proliferation of the MOG autoreactive mouse T cells by delivering either IL10 or TGFb using the assay of FIG. 9. The results show proliferation can be inhibited by the cells. Depending on the ratios of the autoreactive mouse T cells and the therapeutic T cells, similar levels of inhibition can be achieved as MOG reactive regulatory mouse T cells (see for example ratio 1:8) as estimated by the division index. This data shows that engineered human CD4 T cells can inhibit proliferation of MOG autoreactive mouse T cells.

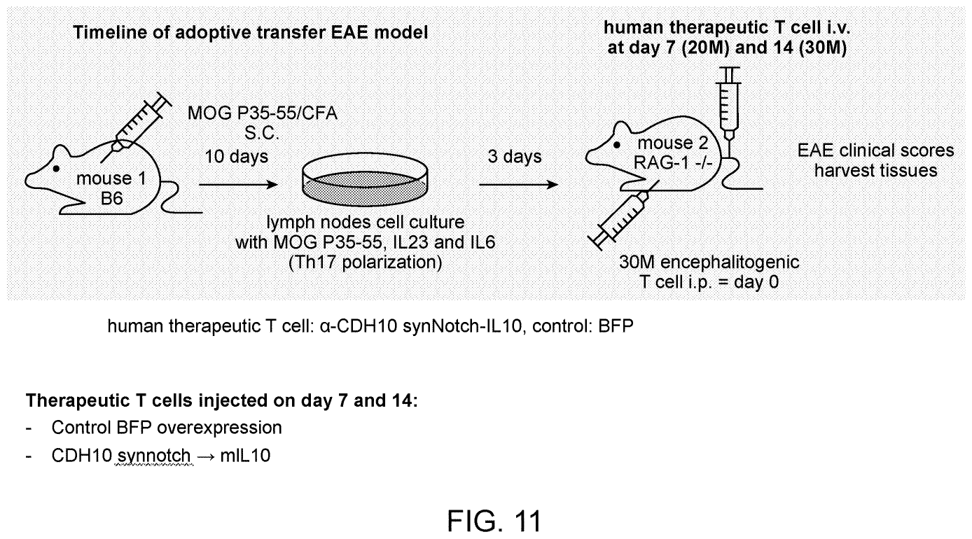

FIG. 11 illustrates the EAE model used in some experiments. Briefly, B6 mice were immunized with the MOG P35-55 peptide, after 10 days their lymph nodes were collected and pieces of spleens and the cells were polarized ex vivo and towards a Th17 phenotype. By adding the MOG P35-55 peptide in that culture the cells that are reactive to the peptide were enriched. These cells are then adoptively transferred into a RAG-1 KO recipient mouse that will develop the disease. At different time points, the therapeutic T cells were injected to test if they can decrease the disease.

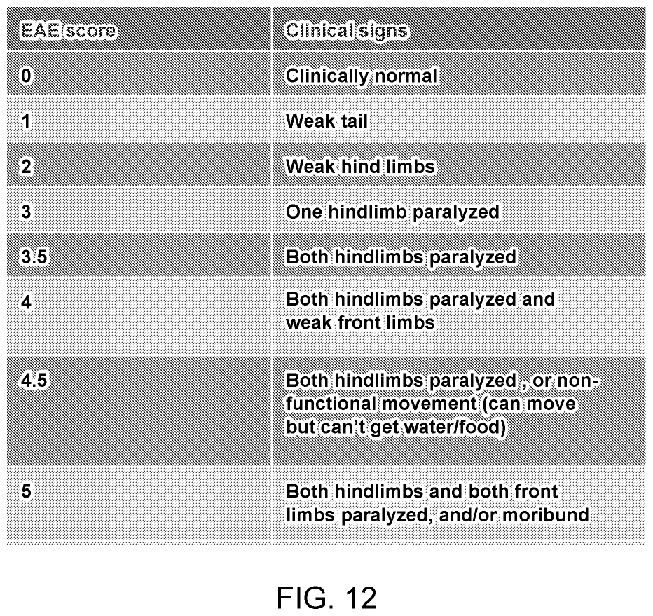

FIG. 12 shows the EAE scoring system, which was used to clinically evaluate the test animals. Briefly, 0 means healthy/normal, 5 is fully paralyzed (moribund/dead).

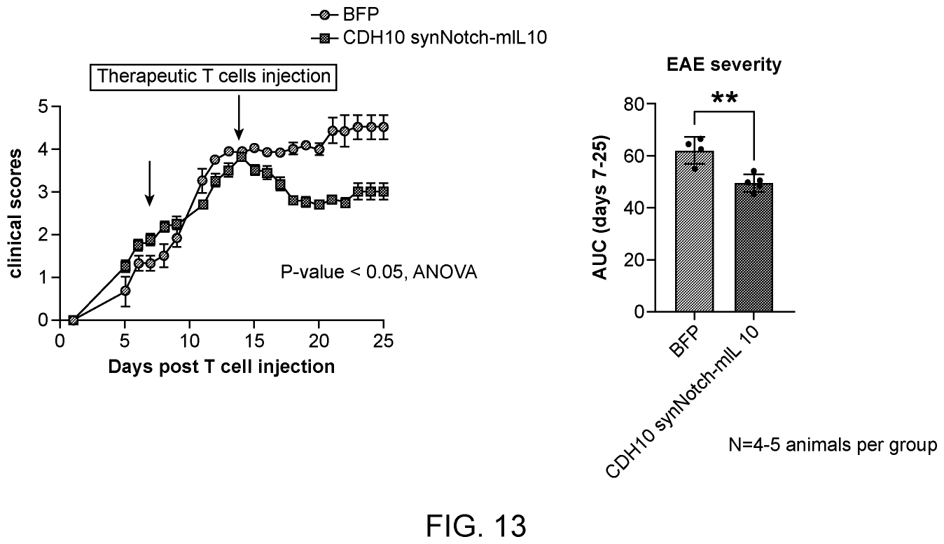

FIG. 13 shows results from an assay designed to test whether the human CDH10 synNotch-IL10 circuit can reduce decrease the severity of the disease. The results show that injections of the CNS specific the human CDH10 synNotch-IL10 circuit were able to decrease the clinical scores of the disease (red line), left graphic. One can also assess the severity of the disease by measuring the AUC (area under the curve) from the time of first treatment to end point (days 7 to 25) for each animal. The results show that the severity of the disease is statistically reduced. This data shows that CNS-specific delivery of mIL10 by primary human CD4 T cells significantly improves EAE scores. Mice treated with the CNS-specific CDH10 synNotch IL10 circuit are visibly healthier than controls (not shown).

Among other things, FIGS. 7-13 shows that:
Human primary CD4 T cells can be engineered to express an anti-proliferative therapy such as IL10 in vitro upon recognition of a CNS-specific marker such as CDH10 (CDH10 synNotch-mIL10 circuit);
CDH10 synNotch-mIL10 primary human T cells inhibit the proliferation of MOG encephalitogenic mouse T cells in vitro;
CDH10 synNotch-mIL10 primary human T cells ameliorate the course of disease in an EAE mouse model of MS;
CDH10 synNotch-mIL10 primary human T cells don't show systemic toxicity.

In a different neuroinflammatory model using human anti-MOG CAR CD4 T cells, it has been shown that that localized/enriched delivery of IL10 extended the survival of the mice. The following synNotch targets were also tested with some positive results: CDH10, CSPG5, PTPRZ1, BCAN, and NrCAM.

### Example 8

### Brain-Targeted Anti-Inflammatory Circuits Targeting CDH10 Ameliorate Autoimmune Encephalomyelitis

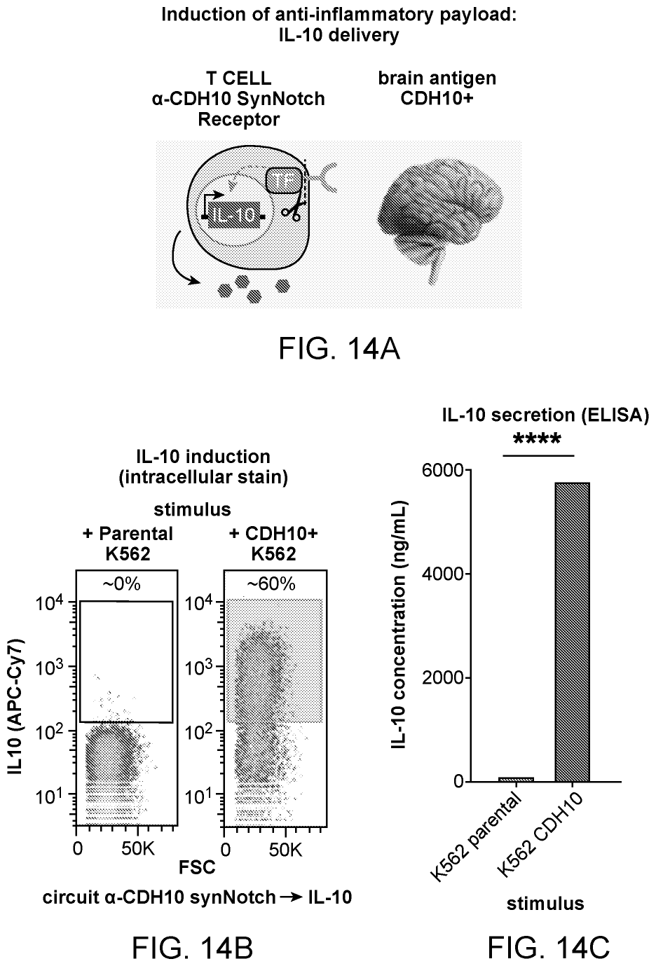

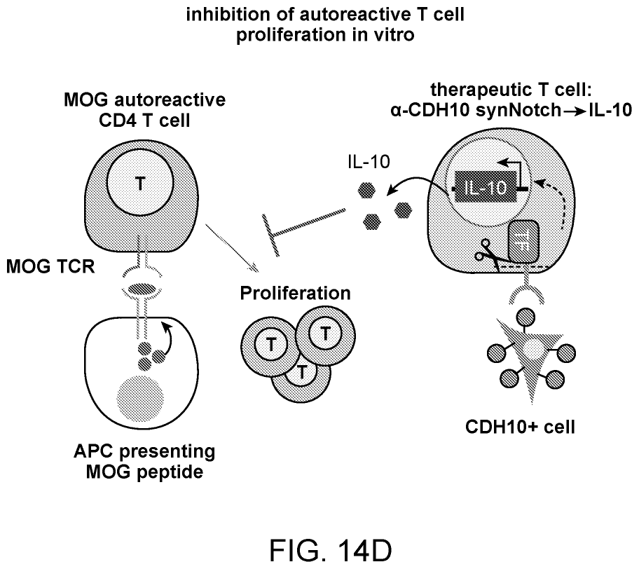

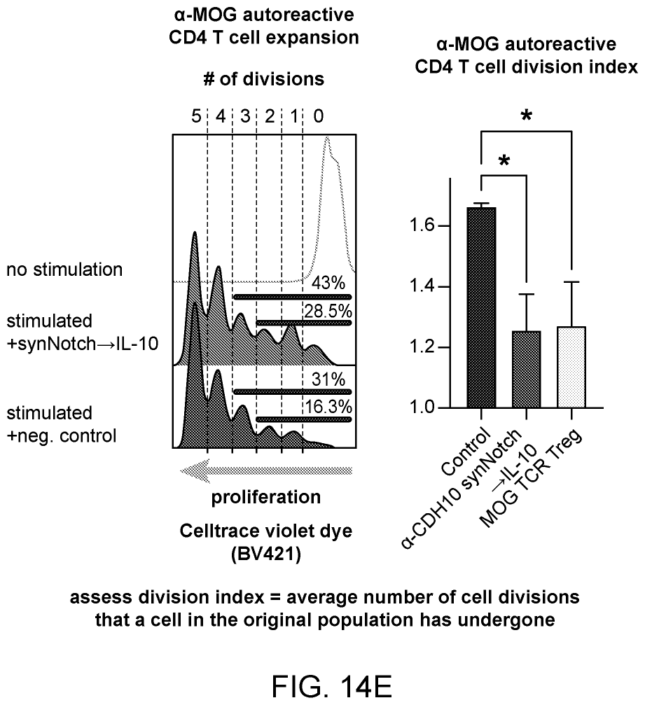

Primary CD4 human T cells were transduced with the brain specific anti-CDH10 synNotch vector and inducible IL10 element vector as described in Choc et al., 2021. T cells were sorted using myc-tag engineered on the synNotch and the constitutive BFP expression marker on the second vector. This circuit is shown in FIG. 14A.

To test the induction of IL10 (FIGS. 14B and 14C), T cells and K562 cells (parental or engineered to overexpressed mouse CDH10) were cocultured at 1:1; 50,000 each in a 1:1 human T cell media: DMEM, 10% FBS for 48 h. Cells were pelleted by centrifugation and the supernatant was collected for ELISA studies using the ELISA MAX Standard Set Mouse IL-10 kit from Biolegend (FIG. 14C). The remaining was resuspended in media containing golgi stop and golgi plug from BD Biosciences and incubated for 4 hours. Cells were then stained with a live/dead stain before being fixed, permeabilized and stained for IL10 using fix/perm and perm/wash reagents from BD Biosciences.

To test the inhibition potential of the CDH10 synNotch-IL10 circuit, an inhibition of proliferation assay was utilized (FIG. 14D). Spleenocytes from 2D2 TCR (TCR^{MOG}) mice were isolated to either serve as antigen presenting cells and were loaded with MOG P35-55 peptide (50 mg/mL) or were stained to sort CD25+ and CD25− CD4 T cells. CD25− CD4 T cells were stained with celltrace violet membrane dye from LifeTechnologies and cocultured with the splenocytes loaded with MOG peptide to induce proliferation, K562 overexpressing CDH10 and human T cells (control or CDH10 SynNotch-IL10) for 5 days, and run by flow cytometry to assess the extent of celltrace violet dilution (FIG. 14E).

This data demonstrates that one can engineer primary human T cells with a brain-specific synNotch circuit to produce anti-inflammatory cytokine IL10 in vitro.

### Example 9

### Engineering Brain-Targeted Immune Suppressor Cells to Recognize BCAN

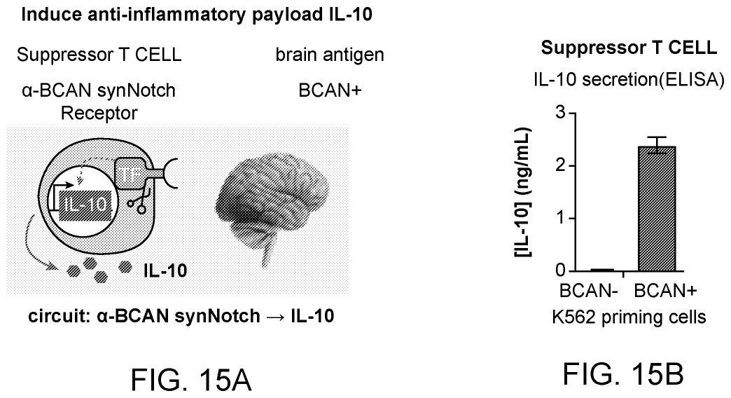

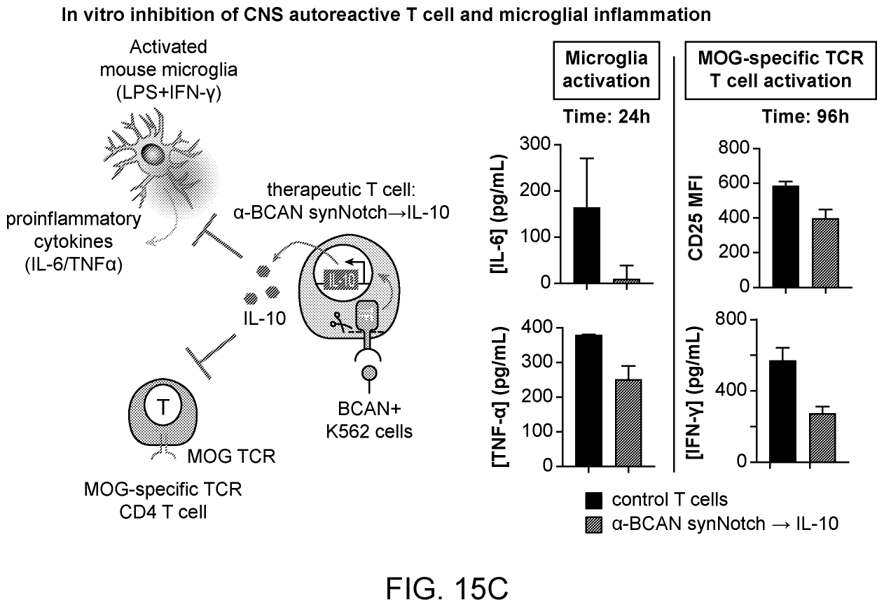

CD4⁺ T cells with the α-BCAN synNotch→IL10 circuit were engineered (FIG. 15A) and it was demonstrated that these cells could effectively produce IL-10 in vitro, but only in the presence of BCAN⁺ K562 cells (FIG. 15B). To evaluate their anti-inflammatory potential, the cells were first tested for their ability to inhibit the activation of both CNS autoreactive T cells and microglia cells in vitro. CNS autoreactive T cells from genetically engineered MOG-specific TCR (2D2) transgenic mice were tested (FIG. 15C), and found that their activation (determined by CD25 staining and IFN-γ secretion) was significantly inhibited by α-BCAN synNotch→IL10 CD4⁺ T cells, in the presence of BCAN⁺ K562 cells (FIG. 15C). Analogously, mouse microglia cells activated with LPS and IFN-γ were tested as previously described (FIG. 15C). The α-BCAN synNotch→IL10 CD4⁺ T cells significantly reduced microglia cell secretion of proinflammatory cytokines such as IL-6 and TNF-α, in the presence of BCAN⁺ K562 cells (FIG. 15C). Thus, the α-BCAN synNotch→IL10 CD4+ T cells, when induced, could exert immunosuppressive activity.

### Example 10

### Brain-Targeted Suppressor Cells that Target BCAN or CDH10 can Ameliorate EAE Disease, a Model for MS

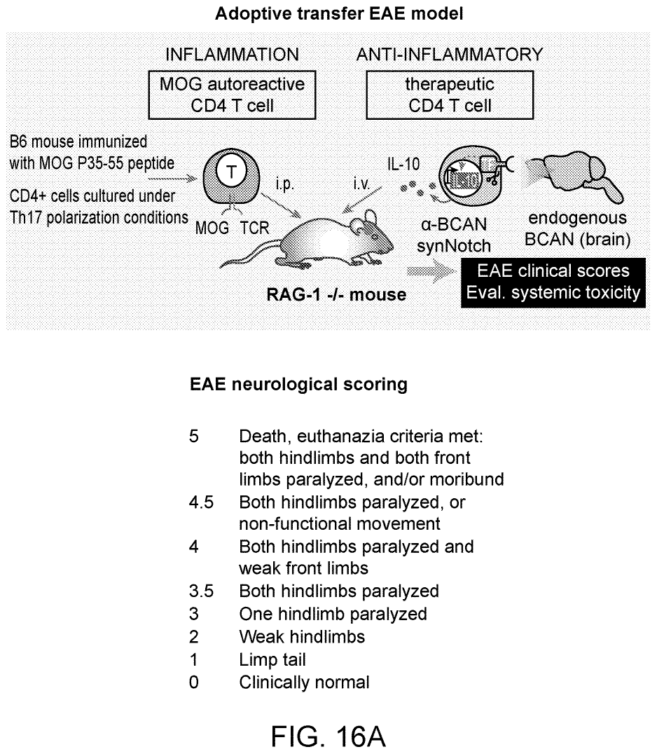

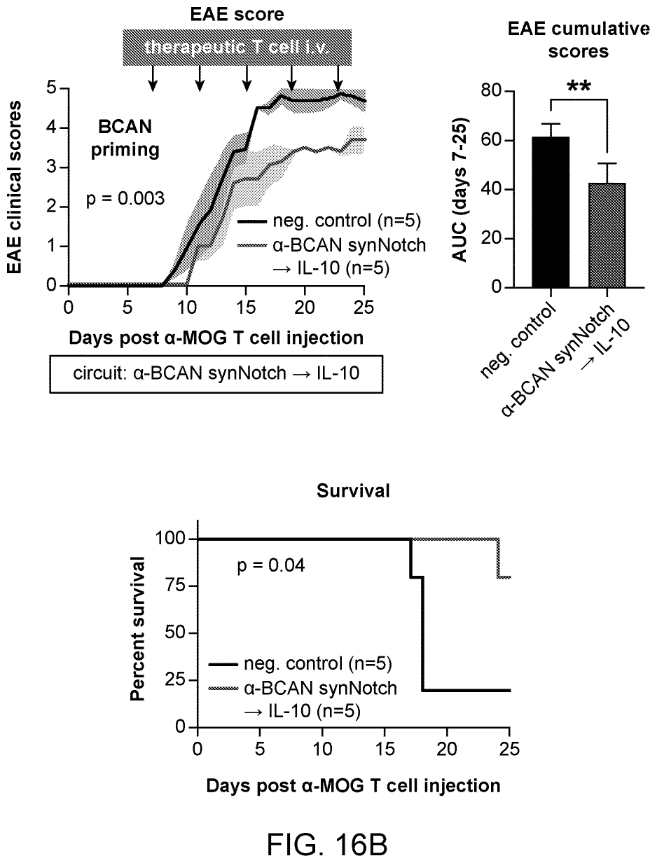

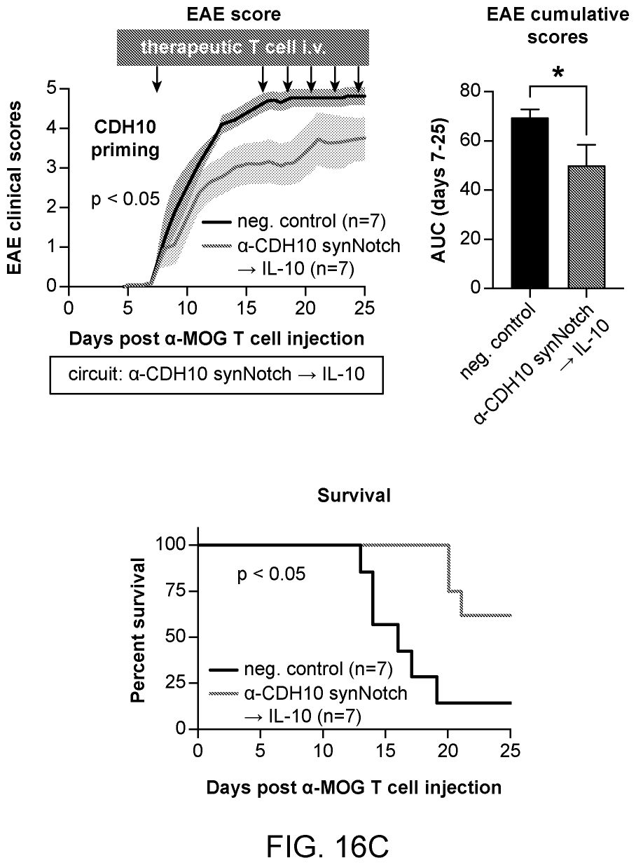

To determine which brain-priming antigens might be best to direct cell therapies against neuroinflammation, published RNA-seq analysis of chronically active MS lesions (compared to healthy CNS tissue of patients) was examined. These data showed maintenance of expression of both BCAN and CDH10 in MS lesions, but a marked decrease in MOG expression (likely caused by demyelination in the disease). Thus, both BCAN and CDH10 are good candidates for priming brain-targeted responses in diseases like MS. To test the efficacy of these brain-targeted suppressor cells in vivo, an EAE model of MS was utilized. An established adoptive transfer model (FIG. 16A) was used, in which pathogenic polyclonal MOG autoreactive Th17 CD4⁺ T cells were generated by direct immunization with MOG peptide. These pathogenic cells were then adoptively transferred into a recipient immunocompromised RAG-1^{-/-} mouse, inducing a severe and often fatal disease, which allowed one to test the efficacy of infusion with clinically relevant human therapeutic T cells. To evaluate the disease progression, the EAE clinical scoring system was used (FIG. 16A).

Adoptive transfer of these α-MOG autoreactive T cells caused highly severe EAE disease in the recipient RAG-1^{-/-} mice, resulting in scores close to the maximum of 5. To test the suppressor cells, the mice were injected i.v. every 4 days with the therapeutic suppressor T cells (or control T cells expressing BFP), starting seven days after adoptive transfer of the disease causing α-MOG autoreactive T cells. The α-BCAN synNotch→IL-10 CD4⁺ T cells improved disease outcome: less severe EAE scores (P=0.003, mixed analysis) were observed, cumulative EAE scores were lowered, and mouse survival increased, relative to mice treated with control T cells (FIG. 16B). Amelioration of EAE disease by the therapeutic cells was supported by the overall increased mobility in the mice treated with α-BCAN synNotch→IL-10 CD4⁺ T cells (control treated mice show increased paralysis). Similarly, treatment with the α-CDH10 synNotch→IL-10 CD4⁺ T cells, also resulted in significantly lower EAE severity scores and increased survival (FIG. 16C). Different dosing regimens of therapeutic T cells provided similar protection. Thus, targeting IL-10 production to the brain, using either BCAN or CDH10 as a priming antigen, resulted in amelioration of EAE symptoms.

The foregoing experiments show that immune cells can be engineered to deliver therapeutic payloads to specific tissues. To engineer immune cells that target the central nervous system (CNS), extracellular CNS-specific antigens were identified, including unique components of the CNS extracellular matrix and neural/oligodendrocyte surface molecules. SynNotch receptors were engineered to detect these antigens and used them to program T cells that induce expression of diverse payloads. CNS-targeted T cells inducing chimeric antigen receptor (CAR) expression efficiently cleared primary and secondary brain tumor xenografts, without killing cross-reactive cells outside the brain. Conversely, CNS-targeted cells delivering immuno-suppressive payloads, like the cytokine IL-10, ameliorated symptoms of a mouse model of neuroinflammatory disease. Thus, CNS-sensing cells provide a flexible new platform to address diverse CNS disorders in a precise anatomically targeted manner.

The foregoing data show that primary human CD4 T cells with a brain-targeted anti-inflammatory circuit that expresses can ameliorate autoimmune encephalomyelitis in vivo in a mouse model of multiple sclerosis, without systemic toxicity.

### REFERENCES

1. V. L. Fcigin et al, Lancet Neurol. 16, 877-897 (2017).
2. G. C. Terstappen et al, Nat. Rev. Drug Discov. 20, 362-383 (2021).
3. J. H. Choe et al, Sci. Transl. Med. 13 (2021).
4. L. Morsut et al, Cell 164, 780-791 (2016).
5. R. Dannenfelser et al, Cell Syst. 11, 215-228.e5 (2020).
6. J. W. Fawcett et al, Nat. Rev. Neurosci. 20, 451-465 (2019).
7. C. Nicholson, et al, Trends Neurosci. 21, 207-215 (1998).
8. K. Bielamowicz et al, Neuro-Oncol. 20, 506-518 (2018).
9. M. Hegde et al, Mol. Ther. J. Am. Soc. Gene Ther. 21, 2087-2101 (2013).
10. J. Wykosky et al, Mol. Cancer Res. 3, 541-551 (2005).
11. L. A. Johnson et al, Blood 114, 535-546 (2009).
12. M. R. Parkhurst et al, Mol. Ther. J. Am. Soc. Gene Ther. 19, 620-626 (2011).
13. R. A. Morgan et al, J. Immunother. Hagerstown Md 1997 36, 133-151 (2013).
14. R. A. Morgan et al, Mol. Ther. J. Am. Soc. Gene Ther. 18, 843-851 (2010).
15. S. E. Syafruddin et al, BMC Cancer 21, 850 (2021).
16. L. Wilkinson et al, Br. J. Radiol. 95, 20211033 (2022).
17. H. Sung et al, Cancer J. Clin. 71, 209-249 (2021).
18. N. Harbeck et al, Nat. Rev. Dis. Primer 5, 1-31 (2019).
19. J. P. Leone et al, Exp. Hematol. Oncol. 4, 33 (2015).
20. R. A. Hernandez-Lopez et al, Science 371, 1166-1171 (2021).
21. D. P. Kodack et al, Proc. Natl. Acad. Sci. U.S.A. 109, E3119-3127 (2012).
22. J. Ni et al, Nat. Med. 22, 723-726 (2016).
23. C. Aversa et al, Breast Edinb. Scotl. 23, 623-628 (2014).
24. N. U. Lin et al, Cancer 113, 2638-2645 (2008).
25. C. K. Anders et al, Cancer 117, 1602-1611 (2011).
26. W. Zhao et al, Oncol. Rep. 40, 759-766 (2018).
27. A. Shvartsur et al, Genes Cancer 6, 84-105 (2015).
28. M. Trerotola et al, Oncogene 32, 222-233 (2013).
29. E. Makhoul et al, Am. J. Clin. Pathol. 152, S37-S37 (2019).
30. D. Liu et al, Mol. Cancer 13, 53 (2014).
31. G. Schett et al, Nat. Commun. 9, 3261 (2018).
32. Y. N. Lamb et al, Drugs 82, 323-334 (2022).
33. P. Maggi et al, eBioMedicine 94 (2023).
34. B. Schreiner et al, Swiss Med. Wkly. 145 (2015).
35. K. Göbel et al, Mult. Scler. J. 24, 432-439 (2018).
36. J. B. Spangler et al, Annu. Rev. Immunol. 33, 139-167 (2015).
37. H. Wiendl et al, Nervenarzt 71, 597-610 (2000).
38. R. D. Huhn et al, Clin. Pharmacol. Ther. 62, 171-180 (1997).
39. A. J. Kastin et al, Mol. Brain Res. 114, 168-171 (2003).
40. L. Li et al, J. Immunol. 153, 3967-3978 (1994).
41. D. J. Cua et al, J. Immunol. 166, 602-608 (2001).
42. E. Bettelli et al, J. Exp. Med. 197, 1073-1081 (2003).
43. N. Gresa-Arribas et al, PLOS ONE 7, e45227 (2012).
44. M. L. Elkjaer et al, Acta Neuropathol. Commun. 7, 205 (2019).
45. P. D. Cravens et al, J. Neuroinflammation 8, 73 (2011).
46. Y. Sonobe et al, Tohoku J. Exp. Med. 213, 329-339 (2007).
47. P. M. Mathisen et al, J. Exp. Med. 186, 159-164 (1997).
48. M. K. Shaw et al, J. Exp. Med. 185, 1711-1714 (1997).
49. A. J. Hou et al, Nat. Rev. Drug Discov. 20, 531-550 (2021).
50. W. A. Lim et al, Cell 168, 724-740 (2017).
51. D. J. Irvine et al, Science 378, 853-858 (2022).
52. C. T. Ellebrecht et al, Science 353, 179-184 (2016).
53. J. Yi et al, Sci. Immunol. 7, eabo0777 (2022).
54. W. A. Lim et al, Science 378, 848-852 (2022).
55. A. Bardia et al, N. Engl. J. Med. 380, 741-751 (2019).
56. A. Shetty et al, Neuroimmunol. Neuroinflammation 1, e22 (2014).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. An engineered immunosuppressive cell comprising a molecular circuit comprising the following components:
(a) a central nervous system (CNS)-specific binding-triggered transcriptional switch (BTTS); and
(b) a nucleic acid encoding an anti-inflammatory payload;
wherein binding of the BTTS to a marker on the surface of a target cell activates expression of the anti-inflammatory payload by the cell.

2. The cell of claim 1, wherein cell is a CD4⁺ T cell, the BTTS is activated by binding to BCAN and the anti-inflammatory payload comprises IL-10 or TGFβ.

3. The cell of claim 1 or 2, wherein the immunosuppressive cell is a T cell, a B cell, a macrophage, or a neutrophil.

4. The cell of any of claims 1-3, wherein the immunosuppressive cell is a CD4⁺ T cell.

5. The cell of any of claims 1-4, wherein the BTTS recognizes a CNS-specific cell surface marker selected from MOG, CDH10, CSPG5, PTPRZ1, BCAN, Neurexin 1b and NrCAM.

6. The cell of any prior claim, wherein the anti-inflammatory payload is:
i an anti-inflammatory cytokine, which may be natural or engineered;
ii an antibody that blocks pro-inflammatory signaling;
iii a soluble receptor that binds to pro-inflammatory cytokine; or
iv a cytokine sink; or
any combination thereof, e.g., i and ii, i and iii, i and iv, ii and iii, ii and iv, iii and iv, i, ii and iii, i, iii and iv, ii, iii and iv or i, ii, iii and iv.

7. The cell of any prior claim, wherein the cell expresses CD25.

8. The cell of any prior claim, wherein the BTTS comprises:
i. an extracellular binding domain that binds to a CNS-specific cell surface marker,
ii. a transmembrane domain; and
ii. an intracellular domain comprising a transcriptional activator, where binding of the extracellular binding domain to the cell surface marker on the surface of another cell induces proteolytic cleavage BTTS to release the transcriptional activator,
wherein the released transcriptional activator induces expression of the payload by the cell.

9. The cell of any prior claim, wherein cell expresses Il-1ra, IL-4, IL-10, IL-11, IL-13, IL-35 and TGF-β, or an engineered variant thereof.

10. The cell of any prior claim, wherein binding of the BTTS to a CNS-specific antigen activates expression of TGFβ1, CD25 and, optionally, IL-10 or a variant thereof.

11. The cell of any prior claim, wherein:
the BTTS is activated by binding to BCAN and the anti-inflammatory payload comprises IL-10;
the BTTS is activated by binding to CDH10 and the anti-inflammatory payload comprises IL-10;
the BTTS is activated by binding to BCAN and the anti-inflammatory payload comprises TGFβ; or
the BTTS is activated by binding to CDH10 and the anti-inflammatory payload comprises TGFβ.

12. The cell of claim 11, wherein the BTTS is activated by binding to BCAN or CDH10 and the anti-inflammatory payload further comprises CD25 and/or IL-2 or a variant thereof.

13. The cell of any prior claim, wherein the cell constitutively expresses CD25.

14. A method of reducing brain inflammation in a subject, comprising:
administering to the subject a cell of any of claims 1-13.

15. The method of claim 14, wherein the subject has an inflammatory brain disease.

16. The method of claim 15, wherein the subject has multiple sclerosis or encephalitis and the method results in a reduction of symptoms.


SEQUENCE LISTING (10 sequences)

SEQ ID NO: 1 - protein, 10 aa, synthetic construct
GRRRRELDPM

SEQ ID NO: 2 - protein, 9 aa, synthetic construct
RQRRELDPM

SEQ ID NO: 3 - protein, 8 aa, synthetic construct
KIEAVKSE

SEQ ID NO: 4 - protein, 8 aa, synthetic construct
KIEAVQSE

SEQ ID NO: 5 - protein, 8 aa, synthetic construct
VGCGVLLS

SEQ ID NO: 6 - protein, 7 aa, synthetic construct
GCGVLLS

SEQ ID NO: 7 - protein, 12 aa, synthetic construct
HEXXHXXGXXHD

SEQ ID NO: 8 - protein, 21 aa, synthetic construct
MALPVTALLLPLALLLHAARP

SEQ ID NO: 9 - protein, 10 aa, synthetic construct
EQKLISEEDL

SEQ ID NO: 10 - DNA, 20 bp, synthetic construct
  source 1..20: mol_type = other DNA
ggagcactgtcctccgaacg